(19) United States
(12) Patent Application Publication
(10) Pub. No.: US 2023/0383269 A1
(43) Pub. Date: Nov. 30, 2023

(54) TETRAMERIC ALPHA/BETA HYDROLASE VARIANTS WITH INCREASED TEMPERATURE STABILITY AND METHODS OF USING AND PRODUCING THEREOF

(71) Applicant: DSM Austria GmbH, Getzerdorf (AT)

(72) Inventors: Andreas HOEBARTNER-GUSSL, Obermeisling (AT); Manuela KILLINGER, Langenlebarn (AT); Gudrun VOGTENTANZ, Krems (AT); Sebastian FRUHAUF, Tulln (AT); Florian KRAINER, Tulln (AT)

(21) Appl. No.: 18/030,883

(22) PCT Filed: Mar. 10, 2021
(86) PCT No.: PCT/EP2021/056026

§ 371 Date: Apr. 7, 2023

(30) Foreign Application Priority Data
Oct. 8, 2020 (EP) 20200918.9

(51) Int. Cl. C12N 9/18; C12N 15/82; A61K 38/46

(52) U.S. Cl. CPC C12N 9/18; C12N 15/8242; A61K 38/465

(57) ABSTRACT

The present invention relates to alpha/beta hydrolase variants with improved properties compared to a parent alpha/beta hydrolase, e.g., having increased temperature stability, methods of using and producing such variants including methods of directing the quaternary structure formation from homo-dimers to homo-tetramers.

This application contains a Sequence Listing in a computer readable form, which is incorporated herein by reference.

### TECHNICAL FIELD

The present invention relates to alpha/beta hydrolase variants with improved properties compared to the parent alpha/beta hydrolase of SEQ ID NO: 1, e.g., having an increased temperature stability, polynucleotides encoding such variants, methods of using and producing such variants including methods of directing the quaternary structure formation from homo-dimers to homo-tetramers. In particular, the present invention relates to methods of using such variants for degrading zearalenone (ZEN).

### BACKGROUND OF THE INVENTION

Mycotoxins are secondary metabolites produced by filamentous fungi. An important representative of mycotoxins is zearalenone (ZEN), which was previously known as F-2 toxin, which is produced by a variety of Fusarium fungi and can be found throughout the world. These fungi infest cultivated plants, among others, such as various types of grain, wherein the fungal infestation usually occurs before the harvest when the growth of the fungi and/or the mycotoxin production may take place before storage or may even take place after harvest, either prior to storage or under improper storage conditions. The Food and Agriculture Organization of the United Nations (FAO) has estimated that 25% of agricultural products throughout the world are contaminated with mycotoxins, thus resulting in substantial economic losses. In an international study spanning 8 years, a total of 19,757 samples was analyzed from January 2004 to December 2011; 72% of them testing positive for at least one mycotoxin, 39% were found to be co-contaminated, and 37% testing positive for ZEN (Schatzmayr and Streit (2013) ‘Global occurrence of mycotoxins in the food and feed chain: Facts and figures.’ World Mycotoxin Journal 6(3):213-222). ZEN has been found in all regions of the world and in all types of grain and feed crops tested, such as corn, soy flour, wheat, wheat bran, DDGS (dried distillers grains with solubles) as well as in finished animal feed mixtures with an incidence of up to 100%.

ZEN binds to the estrogen receptor and can cause hormonal disruptions, being absorbed immediately after oral ingestion and converted by mammals into the two stereoisomeric metabolites α-zearalenol (α-ZEL) and/or β-zearalenol β-ZEL). For example, α-ZEL, but also α-zearalanol (α-ZAL) and/or zearalanone (ZAN), have a much stronger estrogenic effect than ZEN. Although conjugated ZEN derivatives have a lower estrogenic activity than ZEN itself, ZEN can be released again from these conjugated ZEN derivatives in the digestive tract and thereby regain its full estrogenic activity.

ZEN has an oral LD50 of up to 20000 mg/kg body weight, subacute and/or sub-chronic toxic effects such as teratogenic, carcinogenic, estrogenic and immunosuppressant effects may occur in animals or humans with prolonged exposure. Feed contaminated with ZEN leads to developmental disorders in mammalian animals. Pigs and particularly piglets are extremely sensitive to ZEN. ZEN concentrations of more than 0.5 ppm in feed result in developmental disorders, and concentrations of more than 1.5 ppm can result in hyper-estrogenicity in pigs. In cattle, concentrations of 12 ppm ZEN can cause spontaneous abortions.

Since ZEN is absorbed rapidly through the mucous membranes, in particular through the gastric mucosa as well as the oral mucosa, immediate and quantitative deactivation is essential. Already 30 minutes after oral administration, ZEN can be detected in the bloodstream. Because of the harmful effects of ZEN, the European Union has binding upper limits for ZEN in foodstuffs as well as recommendations for upper limits for ZEN in animal feed products (EC No. 1881/2006).

The primary strategy for reducing ZEN contamination in foods and animal feed products is to restrict the growth of fungi, for example, by maintaining “good agricultural practice”. This includes, among other things, ensuring that the seed is free of pests and fungal infestation or that agricultural waste products are removed from the field promptly. In addition, fungal growth in the field can be reduced by the use of fungicides. After the harvest, the harvested material should be stored at a residual moisture level of less than 15% and at a low temperature to prevent the growth of fungi. Likewise, material contaminated by fungal infestation should be removed before further processing. Despite this long list of preventive measures, even in regions with the highest agricultural standards such as North America and Central Europe, up to 37% of the tested corn samples were found contaminated with ZEN in the years 2004 to 2011 (Schatzmayr and Streit (2013)).

Another relevant mycotoxin is ochratoxin A (OTA; also termed e.g. N-{[(3R)-5-chloro-8-hydroxy-3-methyl-1-oxo-3,4-dihydro-1H-2-benzopyran-7-yl]carbonyl}-L-phenylalanine, (-)-N-((5-chloro-8-hydroxy-3-methyl-1-oxo-7-isochromanyl)carbonyl)-3-phenylalanine, (2S)-2-{[(3R)-5-chloro-8-hydroxy-3-methyl-1-oxo-3,4-dihydro-1H-2-benzopyran-7-carbonyl]amino}-3-phenylpropanoic acid, (R)—N-((5-chloro-3,4-dihydro-8-hydroxy-3-methyl-1-oxo-1H-2-benzopyran-7-yl)carbonyl)phenylalanine, N-(((3R)-5-chloro-8-hydroxy-3-methyl-1-oxo-7-isochromanyl)carbonyl)-3-phenyl-L-alanine, N-[(3R)-5-chloro-8-hydroxy-3-methyl-1-oxo-3,4-dihydro-1H-2-benzopyran-7-carbonyl]-L-phenylalanine, N-{[(3R)-5-chloro-8-hydroxy-3-methyl-1-oxo-3,4-dihydro-1H-isochromen-7-yl]carbonyl}-L-phenylalanine), CAS No. 303-47-9, is abundantly produced by fungi and the most toxic member of the ochratoxins group comprising inter alia ochratoxin B, ochratoxin C etc. In particular, OTA imposes serious concerns for food and feed safety due to severe adverse effects on humans and animals, including nephrotoxicity, immunotoxicity and carcinogenicity (Carballo et al., 2019; Malier et al., 2016). In this respect, OTA has been classified by the International Agency of Research on Cancer (IARC) in Group 2B as possible human carcinogen (IARC, 2012).

In order to counteract the above described problems and defects, there was a need to develop α/β-hydrolases variants capable of detoxifying ZEN at higher temperatures and suited for, e.g., use as a food or feed additive or a food or feed product.

The present invention was made in view of the prior art outlined above. The objective of the present invention can therefore be inter alia formulated as to provide improved means and methods to detoxify ZEN (e.g., inter alia provide compositions additionally capable of detoxifying other relevant mycotoxins).

The present application satisfies this demand by the provision of the variants, compositions and methods based thereon described herein below, characterized in the claims and illustrated by the appended Examples and Figures.

### SUMMARY OF THE INVENTION

The present invention relates to a variant of a parent alpha/beta hydrolase, the variant comprising a substitution at one or more positions corresponding to positions: 167, 168, 174, 218, 155, 175, 179, 182, 183, 186, 187, 268, 306, 10, 17, 28, 39, 47, 50, 57, 58, 59, 63, 65, 69, 126, 139, 225, 230, 245, 251, 281, 287, 289, 293 and 304 of SEQ ID NO: 1 (preferably using the numbering of SEQ ID NO: 1), wherein said variant has alpha/beta hydrolase activity and wherein the variant is a polypeptide having at least 71% (preferably: at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%), but less than 100% sequence identity to the polypeptide of SEQ ID NO: 1, the variant having an increased temperature stability compared to the parent alpha/beta hydrolase.

The present invention further relates to a method for degrading zearalenone and/or a derivative thereof, comprising: (a) providing one or more of the following: the variant and/or the parent alpha/beta hydrolase of the present invention, preferably said parent alpha/beta hydrolase having at least 95% sequence identity to the polypeptide of SEQ ID NO: 1; (b) contacting one or more polypeptides from (a) with zearalenone and/or a derivative thereof (e.g., forming an enzyme-substrate mixture).

The present application satisfies this demand by the provision of the variants and compositions described herein below, characterized in the claims and illustrated by the appended Examples and Figures.

### OVERVIEW OF THE SEQUENCE LISTING

SEQ ID NO: 1 is the parent alpha/beta hydrolase amino acid sequence.

SEQ ID NOs: 2-35 are the variant alpha/beta hydrolase amino acid sequences.

SEQ ID NO: 36 is a consensus amino acid motif 1.

SEQ ID NO: 37 is a consensus amino acid motif 2.

SEQ ID NO: 38 is a consensus amino acid motif 3.

SEQ ID NO: 39 is a consensus amino acid motif 4.

### DETAILED DESCRIPTION OF THE INVENTION

### Definitions

As referred herein “EC numbers” (Enzyme Commission numbers) may be used to refer to enzymatic activity according to the Enzyme nomenclature database, Release of Feb. 26, 2020 (e.g., available at https://enzyme.expasy.org/). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively.

As described herein references can be made to UniProtKB Accession Numbers (http://www.uniprot.org/, e.g., as available in UniProtKB release 2020_06 published Dec. 2, 2020)

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is effected e.g. by post-translational modifications like glycosylation, acetylation, phosphorylation and the like. Such modifications are well known in the art.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the no-brief option) is used as the percent identity and is calculated as follows:

(Identical Residues×100)/(Length of Alignment−Total Number of Gaps in Alignment).

Alternatively, the parameters used may be gap open penalty of 10, gap extension penalty of 0.5, and the EDNAFULL (EMBOSS version of NCBI NUC4.4) substitution matrix.

The output of Needle labeled “longest identity” (obtained using the no-brief option) is used as the percent identity and is calculated as follows:

(Identical Deoxyribonucleotides×100)/(Length of Alignment−Total Number of Gaps in Alignment).

Expression: The term “expression” includes any step involved in the production of a variant (polypeptide) including, but not limited to, transcription, post-transcriptional modification, translation, post-translational modification, and secretion.

Expression vector: The term “expression vector” may refer to a linear or circular DNA molecule that comprises a polynucleotide encoding a variant (polypeptide) and is operably linked to control sequences that provide for its expression.

Fragment: The term “fragment” may refer to a polypeptide having one or more (e.g., several) amino acids absent from the amino and/or carboxyl terminus of a mature polypeptide; wherein the fragment still has an activity as described herein (e.g., alpha/beta hydrolase activity, e.g., having e.g. hydrolyzing ZEN to HZEN).

The term “EC:3.1.1.-” as used herein may mean “zearalenone hydrolase” (can also be referred to as “zearalenone lactonase”), e.g., an enzyme capable of hydrolyzing ZEN to HZEN.

Host cell: The term “host cell” may refer to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Nucleic acid construct: The term “nucleic acid construct” may refer to a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” may refer to a configuration in which a control sequence is placed at an appropriate position relative to the coding sequence of a polynucleotide such that the control sequence directs expression of the coding sequence.

Control sequences: The term “control sequences” as used herein may refer to nucleic acid sequences necessary for expression of a polynucleotide encoding a variant (polynucleotide) of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide of the present invention.

As used herein, the term “corresponding to” may refer to a way of determining the specific amino acid of a sequence wherein reference is made to a specific amino acid sequence (e.g., US2020071638). E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Identification of the corresponding amino acid residue in another alpha/beta hydrolase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 51 1-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

For purposes of the present invention, the mature polypeptide disclosed in SEQ ID NO: 1 is used to determine the corresponding amino acid residue in another alpha/beta hydrolase. The amino acid sequence of another alpha/beta hydrolase is aligned with the mature polypeptide disclosed as SEQ ID NO: 1, and based on the alignment, the amino acid position number corresponding to any amino acid residue in the mature polypeptide disclosed as SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

The term “position” when used in accordance with the present invention may refer to a position of an amino acid within an amino acid sequence depicted herein. The term “corresponding” in this context may include that a position is not only determined by the number of the preceding nucleotides/amino acids.

As used herein, “silent” mutations mean base substitutions within a nucleic acid sequence which do not change the amino acid sequence encoded by the nucleic acid sequence. “Conservative or equivalent” substitutions (or mutations) mean substitutions as listed as “Exemplary Substitutions” in Table 1 below. “Highly conservative” substitutions as used herein mean substitutions as shown under the heading “Preferred Substitutions” in Table 1 below.

TABLE 1
Amino acid substitutions

| Original | Exemplary Substitutions | Preferred Substitutions |
|---|---|---|
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |

Variant: The term “variant” may refer to a polypeptide having specific activity as described herein comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of Asn (N) at position 167 with Thr (T) is designated as “N167T” or “Asn167Thr”. Multiple mutations can be separated by addition marks (“+”) or (“,”) e.g., “N167T+F168Y+S174C+F218Y;” or “N167T, F168Y, S174C, F218Y;”, representing multiple substitutions at given positions. In the Examples of the present application, multiple mutations can be separated by comma, e.g., N167T, F168Y, S174C, F218Y. Furthermore, “X” or “Xaa” as used herein may mean any amino acid (e.g., as depicted in Table 1 above). Accordingly, “X167T” as used herein may mean substitution of any amino acid in position 167 with T (Thr). In case where the original amino acid residue may be any amino acid residue, a short hand notation may also be used indicating only the position and substituted amino acid. Accordingly, “X” or “Xaa” may be omitted in designating substitutions, e.g., “167T” designation may be used meaning a substitution of any amino acid in position 167 with T (Thr). Furthermore, “X167G,A,S,C,U,I,L,V,T” as used herein may mean substitution of any amino acid in position 167 with any one of G, A, S, C, U, I, L, V or T. In case where the substituting amino acid residue may be any amino acid residue, a short hand notation may also be used indicating only the original amino acid and its position, e.g., “N167”.

As used herein the term “transgenic” may refer to an organism whose genome has been altered by the incorporation of foreign genetic material or additional copies of native genetic material, e.g. by transformation or recombination (e.g., U.S. Pat. No. 7,410,800B2). The transgenic organism may be a plant, mammal, fungus, bacterium or virus. As used herein “transgenic plant, seed or pollen grain” may refer to a plant, seed or pollen grain or progeny plant, seed or pollen grain of any subsequent generation derived therefrom, wherein the DNA of the plant, seed or pollen grain or progeny thereof contains an introduced exogenous DNA not originally present in a non-transgenic plant, seed or pollen grain of the same strain. The transgenic plant, seed or pollen grain may additionally contain sequences which are native to the plant being transformed, but wherein the exogenous DNA has been altered in order to alter the level or pattern of expression of the coding sequence.

The term “recovering the variant” may refer to purifying the variant, e.g., from a bacterial lysate, e.g., by affinity purification.

The term “foodstuff” may refer to a substance having a food value.

The term “fodder” may refer to a substance fed to domestic animals.

The term “feed” may refer to a substance used as food for livestock.

The term “additive” may refer to a compound or substance added to another product or substance, e.g., in a small amount, to effect a desired property and/or characteristics.

The term “prebiotic” may refer to a compound or substance capable of inducing the growth and/or activity of beneficial microorganisms.

The term “detoxifying agent” may refer to a compound or substance capable of reducing- and/or inhibiting toxicity.

The term “nutritional supplement” may refer to a compound or substance capable to support the nutritional content of the diet, e.g., vitamins and minerals.

The term “intermediate” may refer to a compound or substance produced during (the process (e.g., during an intermediate stage of the process) of obtaining an end-product of the present invention, e.g., foodstuff, fodder, fodder; feed, additive (e.g., foodstuff-, fodder- or feed additive), detoxifying agent, nutritional supplement or prebiotic of the present invention.

The term “phycophytic substance” may refer to a substance derived from a sea algae species.

Amino acid motif: The term “amino acid motif” or “the motif” as used herein may refer to a specifically defined amino acid stretch of a polypeptide. Thus, an amino acid motif of the prevent invention may relate to a short sequence of amino acids within a given polypeptide.

The term “OTA derivative” as used herein may refer to a compound or substance having the following chemical structure:

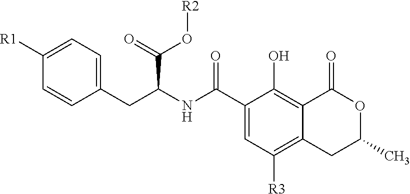

In the molecular structure of an OTA derivative, R1, R2 and R3 may be any atom or group of atoms. In particular, R1 is selected from the group consisting of H and OH, R2 is selected from the group consisting of H and CH2-CH3, and R3 is selected from the group consisting of H and Cl. An OTA derivative may be ochratoxin B, wherein R1 is H, R2 is H, and R3 is H; ochratoxin C, wherein R1 is H, R2 is CH2-CH3, and R3 is Cl; or ochratoxin TA, wherein R1 is OH, R2 is H, and R3 is Cl. In particular, the term “OTA derivative” may refer to a compound or substance selected from the group consisting of: ochratoxin B and ochratoxin C.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

A wildtype patent alpha/beta hydrolase of SEQ ID NO: 1 capable of degrading ZEN to non-toxic HZEN exhibits a low melting point (Tm=50° C.) and has a dimeric homo-dimeric quaternary structure. Since high enzyme stability is a critical point in the production and application of industrial enzymatic products, there was a need to develop further improved α/β-hydrolases variants capable of detoxifying ZEN at higher temperatures and inter alia suitable for use as a food or feed additive or a food or feed product.

In the course of the present invention specific amino acid substitutions were identified in the parent alpha/beta hydrolase having SEQ ID NO: 1 directing the quaternary structure formation of from homo-dimers to homo-tetramers and thus inter alia increasing temperature stability. The present invention thus relates to the use of improved α/β-hydrolase variants described herein for degrading zearalenone (ZEN).

ZEN (CAS Reg. #17924-92-4, (4S,12E)-16,18-dihydroxy-4-methyl-3-oxabicyclo[12.4.0]octadeca-1(14),12,15,17-tetraene-2,8-dione) is a nonsteroidal estrogenic macrocyclic lactone with the following structural formula, synthesized by way of the polyketide metabolic pathway:

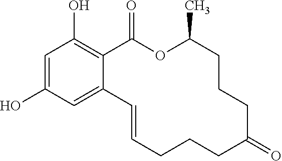 [SMILES: C[C@H]1CCCC(=O)CCC/C=C/c2cc(O)cc(O)c2C(=O)O1]

However, a variety of ZEN derivatives also occurs in nature and may be formed by enzymatic or chemical modifications of ZEN. Examples include glycosidic ZEN conjugates or those containing sulfate, formed by fungi, plants or a mammalian metabolism as well as ZEN metabolites formed in the human or animal organism, among others. ZEN derivatives are understood below to be ZEN conjugates or ZEN metabolites that occur naturally or are synthesized by chemical or biochemical synthesis but in particular α-zearalenol (α-ZEL; CAS Reg. #36455-72-8; (2E,7R,11S)-7,15,17-trihydroxy-11-methyl-12-oxabicyclo[12.4.0]-octadeca-1(18),2,14,16-tetraen-13-one), R-zearalenol (β-ZEL; CAS Reg. #71030-11-0; (2E,7S,11S)-7,15,17-trihydroxy-11-methyl-12-oxabicyclo[12.4.0]octadeca-1(18),2,14,16-tetraen-13-one), α-zearalanol (α-ZAL; CAS Reg. #26538-44-3; (7R,11S)-7,15,17-trihydroxy-11-methyl-12-oxabicyclo[12.4.0]octadeca-1(18),14,16-trien-13-one), R-zearalanol (R-ZAL; CAS Reg. #42422-68-4; (7S,11S)-7,15,17-trihydroxy-11-methyl-12-oxabicyclo[12.4.0]-octadeca-1(14),15,17-trien-13-one), zearalenone 14-sulfate (Z14S; [(2E,11S)-15-hydroxy-11-methyl-7,13-dioxo-12-oxabicyclo[12.4.0]octadeca-1(18),2,14,16-tetraen-17-yl] hydrogen sulfate), zearalenone-14-glycoside (Z14G; (2E,11S)-15-hydroxy-11-methyl-17-[(3R,4S,5S,6R)-3,4,5-trihydroxy-6-(hydroxymethyl)-tetrahydropyran-2-yl]oxy-12-oxabicyclo[12.4.0]octadeca 1(18)2,14,16-tetraene-7,13-dione) as well as zearalanone (ZAN; CAS Reg. #5975-78-0; (11S)-15,17-dihydroxy-11-methyl-12-oxabicyclo-[12.4.0]octadeca-1(18),14,16-triene-7,13-dione).

ZEN as well as ZEN derivatives, in particular α-ZEL, β-ZEL, Z14S, α-ZAL, β-ZAL, Z14G and ZAN can also be detected in processed foods and animal feed products, such as bread or beer because of their high chemical and physical stability.

Hydrolysis of ZEN and ZEN derivatives succeeds with any of the polypeptides of the sequence ID numbers 1 to 35. Hydrolysis of ZEN or its derivatives is believed to occur at the ester group according to the following reaction mechanism:

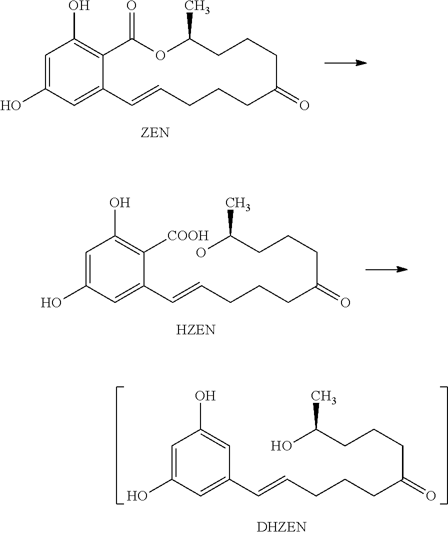

The hydrolysis of ZEN to form nontoxic hydrolyzed zearalenone (HZEN) and/or hydrolyzed ZEN derivatives can take place by the α/β-hydrolases of the present invention. The further decarboxylation of HZEN to decarboxylated hydrolyzed ZEN (DHZEN) and/or decarboxylated hydrolyzed ZEN derivatives is believed to occur spontaneously.

The α/β-hydrolases and variants described herein can be capable of and suitable for degrading ZEN. For example, the α/β-hydrolase, can be suitable for cofactor-free hydrolytic cleavage of the ester group of ZEN and/or its derivatives (e.g., hydrolysis is dependent on H₂O).

ZEN degradation may be measured as follows: a zearalenone degradation assay buffer (118.5 mM NaCl, 8.55 mM acetic acid, 14.9 mM acetate, 0.1 mg/ml BSA, pH 5.0, (Jantratid, E., Janssen, N., Reppas, C. & Dressman, J. B. (2008): Dissolution media simulating conditions in the proximal human gastrointestinal tract: An update. Pharmaceutical Research 25 (7): 1663-1576), containing 0.1 mg/ml bovine serum albumin and 0.52 ppm zearalenone as substrate) is prepared, pre-warmed at 37.0° C. and 960 μl aliquots of assay buffer are transferred into the reaction tubes of a 96 deep well plate. The plate is sealed with a movable lid and kept at 37.0° C. in a DWP-thermo-shaker until the start of the zearalenone degradation assay. Enzyme working solutions are prepared with the purified zearalenone-cleaving polypeptides by dilution in sample buffer (Teorell Stenhagen buffer at pH 7.5, containing 0.1 mg/ml bovine serum albumin) to a 25.0-fold higher concentration as analyzed in the final zearalenone degradation assay, with a concentration of zearalenone-cleaving polypeptides to efficiently degrade zearalenone under the given conditions. To start the zearalenone degradation assay, 40.0 μl of the enzyme working solutions is added to the tubes containing the 960.0 μl assay buffer, hereby achieving a final ZEN concentration of 0.5 ppm in the assay reaction. The addition of the enzyme working solution with pH 7.5 does not change the pH 5.0 of the zearalenone degradation reactions. The zearalenone degradation reactions are incubated in the DWP-thermo-shaker under constant shaking at 37.0° C. Immediately after the zearalenone degradation reaction is started, it was mixed by re-suspending with the pipet tip and a 0.0 h sample of 120.0 μl is transferred into a tube of a new PCR plate and seal with a cap. Additional samples are drawn from the zearalenone degradation reaction after several time points (e.g. 5.0, 10.0, 20.0, 30.0, 45.0, 60.0, 90.0 minutes). As soon as a sample is drawn from the degradation reaction, the zearalenone-cleaving polypeptide in this sample is heat-inactivated by incubation for 10.0 minutes at 99.0° C. in a thermo-block (e.g. Thermal Shake lite, 460-029, VWR). Subsequently, the tube is centrifuged (3.0 minutes, room temperature, 2500×g) and 90.0 μl of the supernatant is transferred into a skirted PCR plate (e.g. twin.tec® PCR plate, 0030128648, Eppendorf AG), which is closed with a heat sealing film (e.g. Heat Sealing Film, 0030127838, Eppendof AG). These sample plates are stored at 4.0° C. until HPLC-FLD measurement. Zearalenone concentration is determined by a modified HPLC-FLD method as described by Vekiru et al. (Vekiru et al. (2016) ‘Isolation and characterization of enzymatic zearalenone hydrolysis reaction products’ World Mycotoxin Journal 9:353-363). Analysis is performed on an HPLC-FLD operated at an extinction of 278.0 nm and an emission at 465.0 nm. The retention time of zearalenone is 1.8 min when separation as done on a Kinetex 5 μm EVO C18 100 A 50.0×2.1 mm column (OOB-4633-AN, Phenomenex Inc.) at 40.0° C. by using solvent A: 95.0% acetonitrile+4.9% water+0.1% formic acid and solvent B: 5.0% acetonitrile+94.9% water+0.1% formic acid when using a gradient: 0.0-0.5 minutes 10% phase B, 0.5-2.0 minutes linear increase to 50.0% phase B, then reduced to 10% phase B in 0.1 minutes, which is continued for the total run time of 2.3 minutes. The flow rate is set at 1.5 ml/min and the injection volume to 5.0 μl. A similar acquisition setup is performed for various analysis. Quantification of zearalenone is based on calibration with external standards of zearalenone. The zearalenone concentration (μM) of the taken reaction samples are plotted against the sampling time point. A slope of the linear zearalenone degradation over time is calculated in μmol zearalenone reduction per minute in the enzymatic reaction volume. By considering possible dilutions of the purified zearalenone-cleaving polypeptides and by including these appropriate dilution factors in the calculation, the specific enzymatic activity determined in μmol zearalenone degradation per minute per mg purified enzyme (μmol ZEN/min/mg) is calculated.

In this context it is noted that the term “unit” or “U” refers to the measure of the catalytic activity of an enzyme and is defined as the number of micromoles (μmol) of substrate, i.e. zearalenone in this case, that are reacted or cleaved per minute under defined conditions. By “activity” of an enzyme or polypeptide solution the enzymatic concentration of the enzyme or polypeptide solution is defined, indicated in units per milliliter (U/ml) or in units per liter (U/I) of solution.

In some embodiments the present invention provides a variant of a parent alpha/beta hydrolase, the variant comprising a substitution at one or more positions corresponding to positions: 167, 168, 174, 218, 155, 175, 179, 182, 183, 186, 187, 268, 306, 10, 17, 28, 39, 47, 50, 57, 58, 59, 63, 65, 69, 126, 139, 225, 230, 245, 251, 281, 287, 289, 293 and 304 of SEQ ID NO: 1 (preferably using the numbering of SEQ ID NO: 1), wherein said variant has alpha/beta hydrolase activity and wherein the variant is a polypeptide having at least 71% (preferably: at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%), but less than 100% sequence identity to the polypeptide of SEQ ID NO: 1, preferably said variant having a hydrolase EC: 3.1.1.-activity.

In further embodiments the variant of the present invention having one or more of the following characteristics: (i) an improved property relative to the parent alpha/beta hydrolase, wherein said improved property comprises an increased temperature stability, preferably as compared to SEQ ID NO: 1; (ii) is capable of tetramerization, preferably having a tetrameric quaternary structure, further preferably said tetramerization is a homo-tetramerization; (iii) having a melting point (Tm) greater than 50° C., preferably said Tm in the range from about 51° C. to about 70° C.; (iv) capable of degrading zearalenone and/or derivatives thereof; (v) comprising at least one (preferably at least two, more preferably at least three, most preferably at least four) amino acid motif/s having a sequence identity of at least 80% (preferably at least 83%, more preferably at least 90%) to an amino acid sequence selected from: (a) SEQ ID NO: 36 (QVDLGEX₁X₂MN), wherein X₁ is V or I, preferably V, and wherein X₂ is V or T, preferably V; (b) SEQ ID NO: 37 (EYDPEWX₃RX₄FX₅EGTV), wherein X₃ is G or A, preferably A, and wherein X₄ is A or V, preferably A, and wherein X₅ is F or Y; (c) SEQ ID NO: 38 (MLX₆QVKTPX₇LX₈THH), wherein X₆ is S or G, preferably S, and wherein X₇ is I or V, preferably 1, and wherein X₈ is I or L, preferably I; and (d) SEQ ID NO: 39 (PAX₉LLX₁₀PEQTGSWWSYEX₁₁X₁₂X₁₃GLLX₁₄EX₁₅FHVX₁₆AVDX₁₇RGQGRSX₁₈WTPX₁₉RYS LDNFGNDLVRFIX₂₀LVX₂₁KRPVX₂₂VX₂₃GNSSGGX₂₄LAAWLSAYX₂₅MPGQX₂₆RX₂₇X₂₈LCED X₂₉X₃₀FFASELVPAX₃₁GHSVX₃₂QX₃₃AGPX₃₄FELX₃₅R), wherein X₉ is V or L, preferably L, and wherein X₁₀ is L or I, preferably L, and wherein X₁₁ is P or E, preferably P, and wherein X₁₂ is V or A, preferably V, and wherein X₁₃ is I or M, preferably 1, and wherein X₁₄ is A or S, preferably A, and wherein X₁₅ is S, N or H, preferably S, and wherein X₁₆ is F or Y, preferably F, and wherein X₁₇ is I or L, preferably 1, and wherein X₁₈ is T or S, preferably T, and wherein X₁₉ is K or R, preferably K, and wherein X₂₀ is S, A or N, preferably S, and wherein X₂₁ is V or I, preferably V, and wherein X₂₂ is I or V, preferably 1, and wherein X₂₃ is S or A, preferably S, and wherein X₂₄ is V or L, preferably V, and wherein X₂₅ is A or S, preferably A, and wherein X₂₆ is I or L, preferably 1, and wherein X₂₇ is A or G, preferably A, and wherein X₂₈ is A or V, preferably A, and wherein X₂₉ is T, A or P, preferably T, and wherein X₃₀ is P or A, preferably P, and wherein X₃₁ is Y or H, preferably H, and wherein X₃₂ is L or R, preferably R, and wherein X₃₃ is A or G, preferably A, and wherein X₃₄ is A or V, preferably A, and wherein X₃₅ is Y or F, preferably Y.

In further embodiments the variant of the present invention comprising one or more of the following substitutions:

X167T, X168Y, X174C, X218Y;
X167T;
X167S;
X167A;
X167C;
X167L;
X167T, X174T;
X167T, X174C;
X155K, X167T, X174C, X175A;
X155K, X167T, X174C, X175T;
X167S, X174C;
X167T, X174C, X218Y;
X167T, X168Y, X174C;
X167T, X168Y, X174C, X179E, X182C, X183R, X186R, X187R, X218Y;
X167T, X168Y, X174C, X179E, X182C, X183R, X186R, X187R, X218Y, X268L, X306I;
X10N, X167T, X168Y, X174C, X179E, X182C, X183R, X186R, X187R, X218Y;
X17Y, X59M, X167T, X168Y, X174C, X179E, X182C, X183R, X186R, X187R, X218Y;
X28T, X167T, X168Y, X174C, X179E, X182C, X183R, X186R, X187R, X218Y;
X39R, X167T, X168Y, X174C, X179E, X182C, X183R, X186R, X187R, X218Y;
X47S, X50E, X167T, X168Y, X174C, X179E, X182C, X183R, X186R, X187R, X218Y;
X57E, X58A, X59M, X167T, X168Y, X174C, X179E, X182C, X183R, X186R, X187R,
X218Y;
X63S, X65H, X167T, X168Y, X174C, X179E, X182C, X183R, X186R, X187R, X218Y;
X69Y, X167T, X168Y, X174C, X179E, X182C, X183R, X186R, X187R, X218Y;
X126S, X167T, X168Y, X174C, X179E, X182C, X183R, X186R, X187R, X218Y;
X139P X167T, X168Y, X174C, X179E, X182C, X183R, X186R, X187R, X218Y;
X167T, X168Y, X174C, X179E, X182C, X183R, X186R, X187R, X218Y, X225S;
X167T, X168Y, X174C, X179E, X182C, X183R, X186R, X187R, X218Y X230R;
X167T, X168Y, X174C, X179E, X182C, X183R, X186R, X187R, X218Y, X245V;
X167T, X168Y, X174C, X179E, X182C, X183R, X186R, X187R, X218Y, X251E;
X167T, X168Y, X174C, X179E, X182C, X183R, X186R, X187R, X218Y, X281E;
X167T, X168Y, X174C, X179E, X182C, X183R, X186R, X187R, X218Y, X287T, X289S;
X167T, X168Y, X174C, X179E, X182C, X183R, X186R, X187R, X218Y, X293S;
X167T, X168Y, X174C, X179E, X182C, X183R, X186R, X187R, X218Y, X304R;
X10N, X17Y, X28T, X39R, X57E, X58A, X59M, X63S, X65H, X69Y, X126S, X139P,
X167T, X168Y, X174C, X179E, X182C, X183R, X186R, X187R, X218Y, X225S, X230R,
X281E, X287T, X289S, X293S, X304R;
X155R, X167T, X168Y, X174C, X175V, X179E, X182C, X183R, X186R, X187R, X189H,
X190S, X192V, X202V, X216V, X218Y, X287T, X289S;
X155R, X167T, X168Y, X174C, X175V, X218Y;
X155R, X167T, X168Y, X174C, X175V;
X155R, X167T, X174C, X175V, X218Y;
X155R, X167T, X168Y, X174C, X175V, X179E, X182C, X183R, X186R, X187R, X218Y;
X155R, X167T, X168Y, X174C, X175V, X189H, X190S, X192V, X218Y;
X155R, X167T, X168Y, X174C, X175V, X179E, X182C, X183R, X186R, X187R, X189H,
X190S, X192V, X218Y;
X155R, X167T, X168Y, X174C, X175V, X179E, X182C, X183R, X186R, X187R, X202V,
X218Y;
X155R, X167T, X168Y, X174C, X175V, X179E, X182C, X183R, X186R, X187R, X202V,
X218Y, X268L, X306I;
X34V, X100N, X140A, X155R, X167T, X168Y, X174C, X175V, X179E, X182C, X183R,
X186R, X187R, X202V, X218Y, X225N, X266R, X268L, X269Q, X306I;
X155R, X167T, X168Y, X174C, X175V, X179E, X182C, X183R, X186R, X187R, X218Y,
X287S, X289S;
X155R, X167T, X168Y, X174C, X175V, X179E, X182C, X183R, X186R, X187R, X202V,
X218Y, X287S, X289S;
X155R, X167T, X168Y, X174C, X175V, X179E, X182C, X183R, X186R, X187R, X202V,
X218Y, X268L, X287S, X289S, X306I;
X34V, X100N, X140A, X155R, X167T, X168Y, X174C, X175V, X179E, X182C, X183R,
X186R, X187R, X202V, X218Y, X225N, X266R, X268L, X269Q, X287S, X289S, X306I;

or an equivalent (e.g., conservative) amino acid substitutions thereof (e.g., as depicted in Table 1 herein).

In further embodiments the parent alpha/beta hydrolase of the present invention, preferably having EC:3.1.1.-hydrolase activity, is selected from the group consisting of: (i) a polypeptide having at least 60% (preferably at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, or 100%) sequence identity to the polypeptide of SEQ ID NO: 1; (ii) a fragment of the polypeptide of SEQ ID NO: 1, wherein said fragment has alpha/beta hydrolase activity, preferably an EC:3.1.1.-hydrolase activity.

In further embodiments the variant of the present invention (i) comprises or consists of a substitution/s selected from the group consisting of:

N167T, F168Y, S174C, F218Y;
N167T;
N167S;
N167A;
N167C;
N167L;
N167T, S174T;
N167T, S174C;
R155K, N167T, S174C, V175A;
R155K, N167T, S174C, V175T;
N167S, S174C;
N167T, S174C, F218Y;
N167T, F168Y, S174C;
N167T, F168Y, S174C, D179E, A182C, A183R, K186R, A187R, F218Y;
N167T, F168Y, S174C, D179E, A182C, A183R, K186R, A187R, F218Y, Q268L, V306I;
D10N, N167T, F168Y, S174C, D179E, A182C, A183R, K186R, A187R, F218Y;
F17Y, I59M, N167T, F168Y, S174C, D179E, A182C, A183R, K186R, A187R, F218Y;
V28T, N167T, F168Y, S174C, D179E, A182C, A183R, K186R, A187R, F218Y;
H39R, N167T, F168Y, S174C, D179E, A182C, A183R, K186R, A187R, F218Y;
E47S, G50E, N167T, F168Y, S174C, D179E, A182C, A183R, K186R, A187R, F218Y;
P57E, V58A, I59M, N167T, F168Y, S174C, D179E, A182C, A183R, K186R, A187R,
F218Y;
A63S, S65H, N167T, F168Y, S174C, D179E, A182C, A183R, K186R, A187R, F218Y;
F69Y, N167T, F168Y, S174C, D179E, A182C, A183R, K186R, A187R, F218Y;
A126S, N167T, F168Y, S174C, D179E, A182C, A183R, K186R, A187R, F218Y;
T139P N167T, F168Y, S174C, D179E, A182C, A183R, K186R, A187R, F218Y;
N167T, F168Y, S174C, D179E, A182C, A183R, K186R, A187R, F218Y, H225S;
N167T, F168Y, S174C, D179E, A182C, A183R, K186R, A187R, F218Y, L230R;
N167T, F168Y, S174C, D179E, A182C, A183R, K186R, A187R, F218Y, A245V;
N167T, F168Y, S174C, D179E, A182C, A183R, K186R, A187R, F218Y, H251E;
N167T, F168Y, S174C, D179E, A182C, A183R, K186R, A187R, F218Y, A281E;
N167T, F168Y, S174C, D179E, A182C, A183R, K186R, A187R, F218Y, L287T, M289S;
N167T, F168Y, S174C, D179E, A182C, A183R, K186R, A187R, F218Y, S293S;
N167T, F168Y, S174C, D179E, A182C, A183R, K186R, A187R, F218Y, S304R;
D10N, F17Y, V28T, H39R, P57E, V58A, I59M, A63S, S65H, F69Y, A126S, T139P, N167T,
F168Y, S174C, D179E, A182C, A183R, K186R, A187R, F218Y, H225S, L230R, A281E,
L287T, M289S, F293S, S304R;

and/or (ii) is selected from the group consisting of: SEQ ID NOs: 2-35.

In further embodiments the present invention provides a method for obtaining a variant of a parent alpha/beta hydrolase, preferably having EC:3.1.1.-hydrolase activity, and/or increasing stability of said parent alpha/beta hydrolase, said method comprising: introducing into the parent alpha/beta hydrolase a substitution at one or more positions corresponding to positions 167, 168, 174, 218, 155, 175, 179, 182, 183, 186, 187, 268, 306, 10, 17, 28, 39, 47, 50, 57, 58, 59, 63, 65, 69, 126, 139, 225, 230, 245, 251, 281, 287, 289, 293 and 304 of SEQ ID NO: 1 (preferably using the numbering of SEQ ID NO: 1), wherein the variant has alpha/beta hydrolase activity; and recovering the variant; preferably said parent alpha/beta hydrolase is according to any one of the preceding claims, further preferably said parent alpha/beta hydrolase having at least 95% sequence identity to the polypeptide of SEQ ID NO: 1.

In further embodiments the present invention relates to a polynucleotide encoding the variant of the present invention. In further embodiments the present invention relates to a nucleic acid construct comprising the polynucleotide of the present invention. In further embodiments the present invention relates to a nucleic acid construct comprising the polynucleotide of the present invention. In further embodiments the present invention relates to a recombinant host cell comprising at least one of the following: (i) variant and/or the parent alpha/beta hydrolase of the present invention; (ii) polynucleotide of the present invention; (iii) nucleic acid construct of the present invention; and/or (iv) expression vector of the present invention.

Accordingly, the present invention also concerns nucleic acid molecules encoding for an α/β-hydrolase variants as described herein. The nucleic acid may be introduced or inserted into an expression vector. The term “expression vector” may refer to a nucleic acid molecule construct that is able to express a gene in vivo or in vitro. In particular, it can encompass DNA constructs suitable for transferring the polypeptide-encoding nucleotide sequence into the host cell (e.g., recombinant host cell) so as to be integrated in the genome or freely located in the extrachromosomal space, and to intracellularly express the polypeptide-encoding nucleotide sequence and, optionally, transport the polypeptide out of the cell. The expression vector as described herein may be expressed in a host cell. The term “host cell” may refer to all cells containing either a nucleotide sequence to be expressed, or an expression vector, and which is able to produce an enzyme or a polypeptide according to the invention. In particular, this refers to prokaryotic and/or eukaryotic cells, preferably Pichia pastoris, Escherichia coli, Bacillus subtilis, Streptomyces, Hansenula, Trichoderma, Lactobacillus, Aspergillus, plant cells and/or spores of Bacillus, Trichoderma or Aspergillus. The name P. pastoris used herein is synonymous with the name Komagataella pastoris, P. pastoris being the older and K. pastoris the systematically newer name (Yamada et al. (1995) ‘The Phylogenetic Relationships of Methanol-assimilating Yeasts Based on the Partial Sequences of 18S and 26S Ribosomal RNAs: The Proposal of Komagataella Gen. Nov. (Saccharomycetaceae)’ Bioscience, Biotechnology and Biochemistry, Vol. 59, issue 3, pp. 439-444). Notably, species of Komagataella pastoris have been recently reassigned to be Komagataella phaffii (Kurtzman (2009) “Biotechnological strains of Komagataella (Pichia) pastoris are Komagataella phaffii as determined from multigene sequence analysis.” J Ind Microbiol Biotechnol. 36(11):1435-8). Komagataella phaffii as used herein can e.g. relate to strains Komagataella phaffii CBS 7435, Komagataella phaffii GS115 or Komagataella phaffii JC308.

In further embodiments the present invention relates to a transgenic plant, transgenic seed or transgenic pollen grain comprising one or more (preferably a plurality) of the following: (i) variant and/or parent alpha/beta hydrolase of the present invention; (ii) polynucleotide of the present invention; (iii) nucleic acid construct of the present invention; (iv) expression vector of the present invention; and/or (v) recombinant host cell of the present invention.

In further embodiments the present invention relates to a foodstuff, intermediate foodstuff; fodder, intermediate fodder; feed, intermediate feed; additive (preferably, foodstuff, fodder- or feed additive), intermediate additive (preferably, foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition, and/or mixture/s thereof comprising one or more of the following: (i) variant and/or parent alpha/beta hydrolase of the present invention; preferably said parent alpha/beta hydrolase having at least 95% sequence identity to the polypeptide of SEQ ID NO: 1; (ii) polynucleotide of the present invention; (iii) nucleic acid construct of the present invention; (iv) expression vector of the present invention; (v) recombinant host cell of the present invention; (vi) transgenic plant, transgenic seed and/or transgenic pollen grain of the present invention.

In further embodiments the present invention relates to a composition or kit comprising one or more of the following: (i) variant and/or the parent alpha/beta hydrolase of the present invention, preferably said parent alpha/beta hydrolase having at least 95% sequence identity to the polypeptide of SEQ ID NO: 1; (ii) polynucleotide of the present invention; (iii) nucleic acid construct of the present invention; (iv) expression vector of the present invention; (v) recombinant host cell of the present invention; (vi) transgenic plant, transgenic seed and/or transgenic pollen grain of the present invention; and/or (vii) foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof of the present invention.

Accordingly, the present invention also relates to a composition comprising an a/p-hydrolase variants as described herein. Preferably, the composition can be a food or feed additive or a food or feed product. Methods to prepare such food- and/or feed compositions are known to the skilled person and are inter alia described in WO 99/35240.

In further embodiments the composition or kit of the present invention, further comprising a nutraceutically acceptable carrier (e.g., water) and/or parent alpha/beta hydrolase of the present invention, preferably said parent alpha/beta hydrolase having at least 95% sequence identity to the polypeptide of SEQ ID NO: 1.

In some further embodiments, the composition or kit of the present invention further comprising one or more of the following: (i) one or more further polypeptides capable of detoxifying OTA and/or at least one OTA derivative (e.g., ochratoxin B and/or ochratoxin C), further preferably said further one or more polypeptides belonging to the M20 Peptidase aminoacylase 1-like protein 2-like amidohydrolase subfamily (e.g., M20 Peptidase ACY1L2 amidohydrolase subfamily, e.g., having identifier cd05672 according to the Conserved Domain Database (e.g., https://www.ncbi.nlm.nih.gov/Structure/cdd/cddsrv.cgi?uid=cd05672), and/or having aminopeptidase activity (e.g., EC 3.4.11.10), and/or comprising a carboxypeptidase activity (e.g., carboxypeptidase A and/or B activity, e.g., having EC 3.4.17.1 and/or EC 3.4.17.2 respectively) and/or thermolysin activity (e.g., having EC 3.4.24.27); (ii) one or more further polypeptides capable of detoxifying one or more mycotoxins (e.g., ZEN and/or trichothecene mycotoxin/s such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin 1, B2, G1 or G2; fumonisins such as e.g. fumonisin 1, B2, B3 or B4; polypeptide mycotoxins such as e.g. beauvericin or enniatins; zearalenone; citrinin; patulin; ergot alkaloids such as e.g. ergotamine) and/or one or more plant- and/or bacteria-derived toxins (e.g. endotoxin, etc.), in particular said one or more further polypeptides capable of detoxifying one or more further mycotoxins and/or one or more plant- and/or bacteria-derived toxins, e.g., a fumonisin esterase (e.g. as disclosed in WO 2016/134387 A1) and/or a zearalenone lactonase (e.g. as disclosed in WO 2020/025580 A1) and/or an ergopeptine hydrolase (e.g. as disclosed in WO 2014/056006 A1); (iii) one or more organic absorbents (e.g., live, inactivated, lyophilized, dormant, and/or dead whole-yeast or yeast-derived product such as e.g. yeast cell wall, or yeast oligosaccharides such as e.g. mannan) and/or one or more inorganic absorbents (e.g., diatomaceous earth and/or clay mineral such as e.g. kaolins or kaolinites, smectites such as e.g. montmorillonites, illites or chlorites; in particular bentonite); (iv) one or more live, inactivated, lyophilized and/or dormant microorganisms capable of detoxifying one or more further mycotoxins (e.g., trichothecene mycotoxins such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin 1, B2, G1 or G2; fumonisins such as e.g. fumonisin 1, B2, B3 or B4; polypeptide mycotoxins such as e.g. beauvericin or enniatins; zearalenone; citrinin; patulin; ergot alkaloids such as e.g. ergotamine) and/or one or more plant- or bacteria-derived toxins (e.g. endotoxin, etc.), in particular said microorganism is selected from the group consisting of: Trichosporon and Apiotrichum genera (e.g. as disclosed in WO 03/053161 A1) and the Coriobacteriaceae family (e.g. as disclosed in EP 3 501 526 A1); (v) one or more plant products (e.g., seaweed, preferably seaweed meal; and/or algae, preferably algae meal; and/or thistle, preferably thistle seeds; and/or glycyrrhiza plant preparation, preferably glycyrrhiza meal and/or glycyrrhiza extract e.g. as disclosed in WO 2018/121881 A1); (vi) one or more flavoring compounds (e.g., plant extract e.g. from oregano, thyme, wintergreen, caraway, marjoram, mint, peppermint, anise, orange, lemon, fennel, star anise, clove, cinnamon and/or garlic; and/or essential oil such as e.g. D-limonene, γ-terpinene, p-cymene, 2-carene, linalool oxide, isomenthone, camphor, linalool, terpinen-4-ol, 2-isopropyl-1-methoxy-4-methylbenzene, L-menthol, ethylamine, α-terpineol, β-caryophyllene, D-carvone, methyl salicylate, α-caryophyllene, lavandulyl acetate, caryophyllene oxide, eugenol, thymol and/or carvacrol); (vii) one or more vitamins (e.g. vitamin A, D, E, K, C, B1, B2, B3, B4, B5, B6, B7, B8, B9 and/or B12; in particular vitamin E).

In particular embodiments, the composition or kit of the present invention further comprising one or more of the following: bentonite, fumonisin esterase and/or a zearalenone actonase, a Coriobacteriaceae microorganism (e.g., a microorganism selected from the family Coriobacteriaceae, e.g., https:elpsn.dsmz.de/family/coriobacteriaceae) capable of detoxifying one or more mycotoxins, diatomaceous earth, yeast (in particular, inactivated yeast), seaweed meal, thistle seeds, and one or more flavoring compound.

In some further embodiments, compositions (e.g., exemplary compositions 1-24 as depicted in Table 2 below) or kits (corresponding to exemplary compositions 1-24 as depicted in Table 2 below) of the present invention comprising one or more further components (e.g., in addition to at least one polypeptide according to the present invention). Such further exemplary compositions or kits are explicitly disclosed in Table 2 herein below, which describes embodiments of the present invention.

TABLE 2
Exemplary compositions or kits of the present invention comprising
one or more further components.

| Composition | | | | | | |
|---|---|---|---|---|---|---|
| Number | 1 | 2 | 3 | 4 | 5 | 6 |
| Any variant or | yes | yes | yes | yes | yes | yes |
| polypeptide | | | | | | |
| according to | | | | | | |
| the present | | | | | | |
| invention | | | | | | |
| (e.g., SEQ ID | | | | | | |
| NOs: 2-35) | | | | | | |
| Any suitable | | | | e.g., | e.g., | |
| polypeptide | | | | fumonisin | fumonisin | |
| capable of | | | | esterase, | esterase, | |
| detoxifying | | | | e.g., having | e.g., | |
| one or more | | | | EC: 3.1.1.87 | having EC: | |
| further | | | | enzymatic | 3.1.1.87 | |
| myco-/plant-/ | | | | activity, | enzymatic | |
| bacterial- | | | | e.g., | activity, | |
| toxins | | | | UniProtKB- | e.g., | |
| | | | | D2D3B6 | UniProtKB- | |
| | | | | and/or | D2D3B6 | |
| | | | | variant/s | and/or | |
| | | | | thereof | variant/s | |
| | | | | | thereof | |
| Any suitable | | e.g., | e.g., | e.g., | | |
| organic | | inactivated | dried yeast | yeast | | |
| absorbent | | yeast | | | | |
| Any suitable | e.g., | e.g., | e.g., | e.g., | e.g., | e.g., |
| inorganic | bentonite | zeolite | bentonite- | bentonite | bentonite | bentonite |
| absorbent | | | montmorillonite | | | |
| Any suitable | e.g., | | | e.g., | e.g., | |
| microorganism | Corio- | | | Corio- | Corio- | |
| capable of | bacteriaceae | | | bacteriaceae | bacteriaceae | |
| detoxifying | | | | | | |
| one or more | | | | | | |
| further | | | | | | |
| mycotoxins | | | | | | |
| Any suitable | | | e.g., | e.g., | | |
| plant product | | | algae | thistle seed | | |
| | | | powder | | | |
| Any suitable | | | | e.g., | | |
| vitamin | | | | Vitamin E | | |
| Any suitable | | | | yes | | |
| flavoring | | | | | | |
| compound | | | | | | |
| Composition | | | | | | |
| Number | 7 | 8 | 9 | 10 | 11 | 12 |
| Any variant or | yes | yes | yes | yes | yes | yes |
| polypeptide | | | | | | |
| according to | | | | | | |
| the present | | | | | | |
| invention | | | | | | |
| (e.g., SEQ ID | | | | | | |
| NOs: 2-35) | | | | | | |
| Any | | | e.g., | e.g., | e.g., | e.g., |
| polypeptide | | | fumonisin | fumonisin | fumonisin | fumonisin |
| capable of | | | esterase, | esterase, | esterase, | esterase, |
| detoxifying | | | e.g., having | e.g., having | e.g., | e.g., |
| one or more | | | EC: 3.1.1.87 | EC: 3.1.1.87 | having | having |
| further | | | enzymatic | enzymatic | EC: 3.1.1.87 | EC: 3.1.1.87 |
| myco-/plant-/ | | | activity, | activity, | enzymatic | enzymatic |
| bacteria- | | | e.g., | e.g., | activity, | activity, |
| toxins | | | UniProtKB- | UniProtKB- | e.g., | e.g., |
| | | | D2D3B6 | D2D3B6 | UniProtKB- | UniProtKB- |
| | | | and/or | and/or | D2D3B6 | D2D3B6 |
| | | | variant/s | variant/s | and/or | and/or |
| | | | thereof | thereof | variant/s | variant/s |
| | | | | | thereof | thereof |
| Any suitable | | e.g., | | e.g., | | |
| organic | | inactivated | | Saccharomyces | | |
| absorbent | | yeast | | cerevisiae | | |
| Any suitable | e.g., | e.g., | e.g., | e.g., | e.g., | e.g., |
| inorganic | bentonite | bentonite | bentonite | bentonite | bentonite | bentonite |
| absorbent | | | | | | |
| Any suitable | e.g., | | e.g., | e.g., | e.g., | |
| microorganism | Eubacterium | | Corio- | Eubacterium | Corio- | |
| capable of | sp. | | bacteriaceae | sp. | bacteriaceae | |
| detoxifying | | | | | | |
| one or more | | | | | | |
| further | | | | | | |
| mycotoxin | | | | | | |
| Any suitable | e.g., | e.g., | | e.g., | | |
| plant product | algae | seaweed | | phycophytic | | |
| | extract | meal, | | substances | | |
| | | milk | | | | |
| | | thistle | | | | |
| Any suitable | | | | | | |
| vitamin | | | | | | |
| Any suitable | | | | | | |
| flavoring | | | | | | |
| compound | | | | | | |
| Composition | | | | | | |
| number | 13 | 14 | 15 | 16 | 17 | 18 |
| Any variant or | yes | yes | yes | yes | yes | yes |
| polypeptide | | | | | | |
| according to | | | | | | |
| the present | | | | | | |
| invention | | | | | | |
| (e.g., SEQ ID | | | | | | |
| NOs: 2-35) | | | | | | |
| Any suitable | | | | e.g. | e.g., | |
| polypeptide | | | | fumonisin | fumonisin | |
| capable of | | | | esterase, | esterase, | |
| detoxifying | | | | e.g., having | e.g., | |
| one or more | | | | EC: 3.1.1.87 | having | |
| further myco- | | | | enzymatic | EC: 3.1.1.87 | |
| /plant- | | | | activity, e.g., | enzymatic | |
| /bacteria- | | | | UniProtKB- | activity, | |
| toxins | | | | D2D3B6 | e.g., | |
| | | | | and/or | UniProtKB- | |
| | | | | variant/s | D2D3B6 | |
| | | | | thereof, | and/or | |
| | | | | zearalenone | variant/s | |
| | | | | lactonase, | thereof, | |
| | | | | e.g., | zearalenone | |
| | | | | UniProtKB- | lactonase | |
| | | | | Q8NKB0 | e.g., | |
| | | | | and/or | UniProtKB- | |
| | | | | variant/s | Q8NKBO | |
| | | | | thereof | and/or | |
| | | | | | variant/s | |
| | | | | | thereof | |
| Any suitable | | e.g., | e.g., | e.g., | | |
| organic | | inactivated | dried yeast | yeast | | |
| absorbent | | yeast | | | | |
| Any suitable | e.g., | e.g., | e.g., | e.g., | e.g., | e.g., |
| inorganic | bentonite | zeolite | bentonite- | bentonite | bentonite | bentonite |
| absorbent | | | montmorillonite | | | |
| Any suitable | e.g., | | | e.g., | e.g., | |
| microorganis | Corio- | | | Corio- | Corio- | |
| m capable of | bacteriaceae | | | bacteriaceae | bacteriaceae | |
| detoxifying | | | | | | |
| one or more | | | | | | |
| further | | | | | | |
| mycotoxins | | | | | | |
| Any suitable | | | e.g., | | | |
| plant product | | | algae | | | |
| | | | powder | | | |
| Any suitable | | | | e.g., | | |
| vitamin | | | | Vitamin E | | |
| Any suitable | | | | yes | | |
| flavoring | | | | | | |
| compound | | | | | | |
| Composition | | | | | | |
| number | 19 | 20 | 21 | 22 | 23 | 24 |
| Any variant or | yes | yes | yes | yes | yes | yes |
| polypeptide | | | | | | |
| according to | | | | | | |
| the present | | | | | | |
| invention | | | | | | |
| (e.g., SEQ ID | | | | | | |
| NOs: 2-35) | | | | | | |
| Any | | | fumonisin | fumonisin | fumonisin | fumonisin |
| polypeptide | | | esterase, | esterase, | esterase, | esterase, |
| capable of | | | e.g., having | e.g., having | e.g., | e.g., |
| detoxifying | | | EC: 3.1.1.87 | EC: 3.1.1.87 | having | having |
| one or more | | | enzymatic | enzymatic | EC: 3.1.1.87 | EC: 3.1.1.87 |
| further | | | activity, e.g., | activity, e.g., | enzymatic | enzymatic |
| myco-/plant-/ | | | UniProtKB- | UniProtKB- | activity, | activity, |
| bacteria- | | | D2D3B6 | D2D3B6 | e.g., | e.g., |
| toxins | | | and/or | and/or | UniProtKB- | UniProtKB- |
| | | | variant/s | variant/s | D2D3B6 | D2D3B6 |
| | | | thereof, | thereof, | and/or | and/or |
| | | | zearalenone | zearalenone | variant/s | variant/s |
| | | | lactonase, | lactonase, | thereof, | thereof, |
| | | | e.g., | e.g., | zearalenone | zearalenone |
| | | | UniProtKB- | UniProtKB- | lactonase, | lactonase, |
| | | | Q8NKB0 | Q8NKB0 | e.g., | e.g., |
| | | | and/or | and/or | UniProtKB- | UniProtKB- |
| | | | variant/s | variant/s | Q8NKB0 | Q8NKBO |
| | | | thereof | thereof | and/or | and/or |
| | | | | | variant/s | variant/s |
| | | | | | thereof | thereof |
| Any suitable | | e.g., | | e.g., | | |
| organic | | inactivated | | Saccharomyces | | |
| absorbent | | yeast | | cerevisiae | | |
| Any suitable | e.g., | e.g., | e.g., | e.g., | e.g., | e.g., |
| inorganic | bentonite | bentonite | bentonite | bentonite | bentonite | bentonite |
| absorbent | | | | | | |
| Any suitable | e.g., | | e.g., | e.g., | e.g., | |
| microorganism | Eubacterium | | Corio- | Eubacterium | Corio- | |
| capable of | sp. | | bacteriaceae | sp. | bacteriaceae | |
| detoxifying | | | | | | |
| one or more | | | | | | |
| further | | | | | | |
| mycotoxins | | | | | | |
| Any suitable | e.g., | e.g., | | e.g., | | |
| plant product | algae | seaweed | | phycophytic | | |
| | extract | meal, | | substances | | |
| | | milk | | | | |
| | | thistle | | | | |
| Any suitable | | | | | | |
| vitamin | | | | | | |
| Any suitable | | | | | | |
| flavoring | | | | | | |
| compound | | | | | | |

In further embodiments, the variant, parent alpha/beta hydrolase, polynucleotide, nucleic acid construct, expression vector, recombinant host cell, transgenic plant, transgenic seed, transgenic pollen grain, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof, composition and/or kit of the present invention are suitable for use as a medicament and/or in therapy, preferably for use in the prophylaxis or treatment of a disease, further preferably said parent alpha/beta hydrolase having at least 95% sequence identity to the polypeptide of SEQ ID NO: 1.

In further embodiments, the variant, parent alpha/beta hydrolase, polynucleotide, nucleic acid construct, expression vector, recombinant host cell, transgenic plant, transgenic seed, transgenic pollen grain, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof, composition and/or kit of the present invention, are suitable for use in the prophylaxis or treatment of mycotoxicosis, preferably of hormonal disruption caused by zearalenone or a derivative thereof.

In further embodiments, the present invention relates to use of the variant, parent alpha/beta hydrolase polynucleotide, nucleic acid construct, expression vector, recombinant host cell, transgenic plant, transgenic seed, transgenic pollen grain, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof of the present invention, for/in/as one or more of the following: (i) degradation of zearalenone and/or a derivative thereof; (ii) food, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition; (iii) manufacture/production of one or more of the following: foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition, and/or mixture/s thereof; (iv) manufacture/production of silage, biogas, bioethanol, or sugar, preferably from sugar cane or sugar beets; (v) prevention and/or treatment of mycotoxicosis.

In further embodiments, the present invention provides a method for degrading zearalenone and/or a derivative thereof, comprising: (a) providing one or more of the following: (i) variant and/or the parent alpha/beta hydrolase of the present invention, preferably said parent alpha/beta hydrolase having at least 95% sequence identity to the polypeptide of SEQ ID NO: 1; (ii) polynucleotide of the present invention; (iii) nucleic acid construct of the present invention; (iv) expression vector of the present invention; (v) recombinant host cell of the present invention; (vi) transgenic plant, transgenic seed and/or transgenic pollen grain of the present invention; (vii) foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof of the present invention; and/or (viii) composition or kit of the present invention; (b) contacting one or more from (a) with zearalenone and/or a derivative thereof (e.g., forming an enzyme-substrate mixture).

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The invention is also characterized by the following items:
1. A variant of a parent alpha/beta hydrolase, the variant comprising a substitution at one or more positions corresponding to positions: 167, 168, 174, 218, 155, 175, 179, 182, 183, 186, 187, 268, 306, 10, 17, 28, 39, 47, 50, 57, 58, 59, 63, 65, 69, 126, 139, 225, 230, 245, 251, 281, 287, 289, 293 and 304 of SEQ ID NO: 1 (preferably using the numbering of SEQ ID NO: 1), wherein said variant has alpha/beta hydrolase activity (e.g., zearalenone hydrolyse) and wherein the variant is a polypeptide having at least 71% (preferably: at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%), but less than 100% sequence identity to the polypeptide of SEQ ID NO: 1, preferably said variant having a hydrolase EC:3.1.1.-activity.
2. The variant according to any one of the preceding items, wherein said variant having one or more of the following:
   i) an improved property relative to the parent alpha/beta hydrolase, wherein said improved property comprises an increased temperature stability, preferably as compared to SEQ ID NO: 1;
   ii) is capable of tetramerization, preferably having a tetrameric quaternary structure, further preferably said tetramerization is a homo-tetramerization;
   iii) a melting point (Tm) greater than 50° C., preferably said Tm
   iv) in the range from about 51° C. to about 70° C.;
   v) capable of degrading zearalenone and/or derivatives thereof;
   vi) comprising at least one, preferably at least two, more preferably at least three, most preferably at least four amino acid motif/s having a sequence identity of at least 80%, preferably at least 83%, more preferably at least 90% to an amino acid sequence selected from:
      a) QVDLGEX₁X₂MN (SEQ ID NO: 36), wherein X, is V or I, preferably V, and wherein X₂ is V or T, preferably V;
      b) EYDPEWX₃RX₄FX₅EGTV (SEQ ID NO: 37), wherein X₃ is G or A, preferably A, and wherein X₄ is A or V, preferably A, and wherein X₅ is F or Y;
      c) MLX₆QVKTPX₇LX₈THH (SEQ ID NO: 38), wherein X₆ is S or G, preferably S, and wherein X₇ is I or V, preferably 1, and wherein X₈ is I or L, preferably I; and
      d) PAX₉LLX₁₀PEQTGSWWSYEX₁₁X₁₂X₁₃GLLX₁₄EX₁₅FHVX₁₆AVDX₁₇RGQGR SX₁₈WTPX₁₉RYSLDNFGNDLVRFIX₂₀LVX₂₁KRPVX₂₂VX₂₃GNSSGGX₂₄LAA WLSAYX₂₅MPGQX₂₆RX₂₇X₂₈LCEDX₂₉X₃₀FFASELVPAX₃₁GHSVX₃₂QX₃₃AG PX₃₄FELX₃₅R (SEQ ID NO: 39), wherein X₉ is V or L, preferably L, and wherein X₁₀ is L or I, preferably L, and wherein X₁₁ is P or E, preferably P, and wherein X₁₂ is V or A, preferably V, and wherein X₁₃ is I or M, preferably 1, and wherein X₁₄ is A or S, preferably A, and wherein X₁₅ is S, N or H, preferably S, and wherein X₁₆ is F or Y, preferably F, and wherein X₁₇ is I or L, preferably 1, and wherein X₁₈ is T or S, preferably T, and wherein X₁₉ is K or R, preferably K, and wherein X₂₀ is S, A or N, preferably S, and wherein X₂₁ is V or I, preferably V, and wherein X₂₂ is I or V, preferably 1, and wherein X₂₃ is S or A, preferably S, and wherein X₂₄ is V or L, preferably V, and wherein X₂₅ is A or S, preferably A, and wherein X₂₆ is I or L, preferably 1, and wherein X₂₇ is A or G, preferably A, and wherein X₂₈ is A or V, preferably A, and wherein X₂₉ is T, A or P, preferably T, and wherein X₃₀ is P or A, preferably P, and wherein X₃₁ is Y or H, preferably H, and wherein X₃₂ is L or R, preferably R, and wherein X₃₃ is A or G, preferably A, and wherein X₃₄ is A or V, preferably A, and wherein X₃₅ is Y or F, preferably Y.
3. The variant according any one of the preceding items, wherein the number of substitutions is 1-30, preferably, 1-20 and 1-10, such as 1, 2, 3, 4, 5, 6, 7, 8, 9 or 10 substitutions.
4. The variant according any one of the preceding items, wherein said variant comprising a substitution at one or more positions corresponding to the following positions of SEQ ID NO: 1 (e.g., each row of the tables below represent an individual exemplary combination of features according to the present invention, e.g., specific embodiments of variants):

N167, F168, S174, F218;
N167;
N167, S174;
R155, N167, S174, V175;
N167, S174;
N167, S174, F218;
N167, F168, S174;
N167, F168, S174, D179, A182, A183, K186, A187, F218;
N167, F168, S174, D179, A182, A183, K186, A187, F218, Q268, V306;
D10, N167, F168, S174, D179, A182, A183, K186, A187, F218;
F17, I59, N167, F168, S174, D179, A182, A183, K186, A187, F218;
V28, N167, F168, S174, D179, A182, A183, K186, A187, F218;
H39, N167, F168, S174, D179, A182, A183, K186, A187, F218;
E47, G50, N167, F168, S174, D179, A182, A183, K186, A187, F218;
P57, V58, I59, N167, F168, S174, D179, A182, A183, K186, A187, F218;
A63, S65, N167, F168, S174, D179, A182, A183, K186, A187, F218;
F69, N167, F168, S174, D179, A182, A183, K186, A187, F218;
A126, N167, F168, S174, D179, A182, A183, K186, A187, F218;
T139 N167, F168, S174, D179, A182, A183, K186, A187, F218;
N167, F168, S174, D179, A182, A183, K186, A187, F218, H225;
N167, F168, S174, D179, A182, A183, K186, A187, F218, L230;
N167, F168, S174, D179, A182, A183, K186, A187, F218, A245;
N167, F168, S174, D179, A182, A183, K186, A187, F218, H251;
N167, F168, S174, D179, A182, A183, K186, A187, F218, A281;
N167, F168, S174, D179, A182, A183, K186, A187, F218, L287, M289;
N167, F168, S174, D179, A182, A183, K186, A187, F218, S293;
N167, F168, S174, D179, A182, A183, K186, A187, F218, S304;
D10, F17, V28, H39, P57, V58, I59, A63, S65, F69, A126, T139, N167, F168, S174,
D179, A182, A183, K186, A187, F218, H225, L230, A281, L287, M289, F293, S304;

5. The variant according any one of the preceding items, wherein said variant comprising one or more of the following substitutions (e.g., each row of the tables below represent an individual exemplary combination of features according to the present invention, e.g., specific embodiments of variants):

X167T, X168Y, X174C, X218Y;
X167T;
X167S;
X167A;
X167C;
X167L;
X167T, X174T;
X167T, X174C;
X155K, X167T, X174C, X175A;
X155K, X167T, X174C, X175T;
X167S, X174C;
X167T, X174C, X218Y;
X167T, X168Y, X174C;
X167T, X168Y, X174C, X179E, X182C, X183R, X186R, X187R, X218Y;
X167T, X168Y, X174C, X179E, X182C, X183R, X186R, X187R, X218Y, X268L,
X306I;
X10N, X167T, X168Y, X174C, X179E, X182C, X183R, X186R, X187R, X218Y;
X17Y, X59M, X167T, X168Y, X174C, X179E, X182C, X183R, X186R, X187R,
X218Y;
X28T, X167T, X168Y, X174C, X179E, X182C, X183R, X186R, X187R, X218Y;
X39R, X167T, X168Y, X174C, X179E, X182C, X183R, X186R, X187R, X218Y;
X47S, X50E, X167T, X168Y, X174C, X179E, X182C, X183R, X186R, X187R,
X218Y;
X57E, X58A, X59M, X167T, X168Y, X174C, X179E, X182C, X183R, X186R, X187R,
X218Y;
X63S, X65H, X167T, X168Y, X174C, X179E, X182C, X183R, X186R, X187R,
X218Y;
X69Y, X167T, X168Y, X174C, X179E, X182C, X183R, X186R, X187R, X218Y;
X126S, X167T, X168Y, X174C, X179E, X182C, X183R, X186R, X187R, X218Y;
X139P X167T, X168Y, X174C, X179E, X182C, X183R, X186R, X187R, X218Y;
X167T, X168Y, X174C, X179E, X182C, X183R, X186R, X187R, X218Y, X225S;
X167T, X168Y, X174C, X179E, X182C, X183R, X186R, X187R, X218Y X230R;
X167T, X168Y, X174C, X179E, X182C, X183R, X186R, X187R, X218Y, X245V;
X167T, X168Y, X174C, X179E, X182C, X183R, X186R, X187R, X218Y, X251E;
X167T, X168Y, X174C, X179E, X182C, X183R, X186R, X187R, X218Y, X281E;
X167T, X168Y, X174C, X179E, X182C, X183R, X186R, X187R, X218Y, X287T,
X289S;
X167T, X168Y, X174C, X179E, X182C, X183R, X186R, X187R, X218Y, X293S;
X167T, X168Y, X174C, X179E, X182C, X183R, X186R, X187R, X218Y, X304R;
X10N, X17Y, X28T, X39R, X57E, X58A, X59M, X63S, X65H, X69Y, X126S, X139P,
X167T, X168Y, X174C, X179E, X182C, X183R, X186R, X187R, X218Y, X225S,
X230R, X281E, X287T, X289S, X293S, X304R;

X155R, X167T, X168Y, X174C, X175V, X179E, X182C, X183R, X186R, X187R,
X189H, X190S, X192V, X202V, X216V, X218Y, X287T, X289S;
X155R, X167T, X168Y, X174C, X175V, X218Y;
X155R, X167T, X168Y, X174C, X175V;
X155R, X167T, X174C, X175V, X218Y;
X155R, X167T, X168Y, X174C, X175V, X179E, X182C, X183R, X186R, X187R,
X218Y;
X155R, X167T, X168Y, X174C, X175V, X189H, X190S, X192V, X218Y;
X155R, X167T, X168Y, X174C, X175V, X179E, X182C, X183R, X186R, X187R,
X189H, X190S, X192V, X218Y;
X155R, X167T, X168Y, X174C, X175V, X179E, X182C, X183R, X186R, X187R,
X202V, X218Y;
X155R, X167T, X168Y, X174C, X175V, X179E, X182C, X183R, X186R, X187R,
X202V, X218Y, X268L, X306I;
X34V, X100N, X140A, X155R, X167T, X168Y, X174C, X175V, X179E, X182C,
X183R, X186R, X187R, X202V, X218Y, X225N, X266R, X268L, X269Q, X306I;
X155R, X167T, X168Y, X174C, X175V, X179E, X182C, X183R, X186R, X187R,
X218Y, X287S, X289S;
X155R, X167T, X168Y, X174C, X175V, X179E, X182C, X183R, X186R, X187R,
X202V, X218Y, X287S, X289S;
X155R, X167T, X168Y, X174C, X175V, X179E, X182C, X183R, X186R, X187R,
X202V, X218Y, X268L, X287S, X289S, X306I;
X34V, X100N, X140A, X155R, X167T, X168Y, X174C, X175V, X179E, X182C,
X183R, X186R, X187R, X202V, X218Y, X225N, X266R, X268L, X269Q, X287S,
X289S, X306I;

or equivalent amino acid substitutions thereof.
6. The variant according any one of the preceding items, wherein said variant comprising one or more of the following substitutions:
   X₁₆₇G,A,S,C,U,I,L,V,T;
   X₁₇₄G,A,S,C,U,I,L,V,T;
   X₁₅₅R,H,K;
   X₁₇₅G,A,S,C,U,V,T;
   X₂₁₈Y,F,H; and/or
   X₁₆₈Y, F, H.
7. The variant according any one of the preceding items, wherein said parent alpha/beta hydrolase, preferably having EC:3.1.1.-hydrolase activity, is selected from the group consisting of:
   i) a polypeptide having at least 60% (preferably at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, or 100%) sequence identity to the polypeptide of SEQ ID NO: 1;
   ii) a fragment of the polypeptide of SEQ ID NO: 1, wherein said fragment has alpha/beta hydrolase activity, preferably an EC:3.1.1.-hydrolase activity.
8. The variant according any one of the preceding items, wherein the parent alpha/beta hydrolase comprises or consists of the polypeptide of SEQ ID NO: 1.
9. The variant according any one of preceding items, wherein said variant:
   i) comprises or consists of a substitution/s selected from the group consisting of (e.g., each row of the tables below represent an individual exemplary combination of features according to the present invention, e.g., specific embodiments of variants):

N167T, F168Y, S174C, F218Y;
N167T;
N167S;
N167A;
N167C;
N167L;
N167T, S174T;
N167T, S174C;
R155K, N167T, S174C, V175A;
R155K, N167T, S174C, V175T;
N167S, S174C;
N167T, S174C, F218Y;
N167T, F168Y, S174C;
N167T, F168Y, S174C, D179E, A182C, A183R, K186R, A187R, F218Y;
N167T, F168Y, S174C, D179E, A182C, A183R, K186R, A187R, F218Y, Q268L,
V306I;
D10N, N167T, F168Y, S174C, D179E, A182C, A183R, K186R, A187R, F218Y;
F17Y, I59M, N167T, F168Y, S174C, D179E, A182C, A183R, K186R, A187R,
F218Y;
V28T, N167T, F168Y, S174C, D179E, A182C, A183R, K186R, A187R, F218Y;
H39R, N167T, F168Y, S174C, D179E, A182C, A183R, K186R, A187R, F218Y;
E47S, G50E, N167T, F168Y, S174C, D179E, A182C, A183R, K186R, A187R,
F218Y;
P57E, V58A, I59M, N167T, F168Y, S174C, D179E, A182C, A183R, K186R, A187R,
F218Y;
A63S, S65H, N167T, F168Y, S174C, D179E, A182C, A183R, K186R, A187R,
F218Y;
F69Y, N167T, F168Y, S174C, D179E, A182C, A183R, K186R, A187R, F218Y;
A126S, N167T, F168Y, S174C, D179E, A182C, A183R, K186R, A187R, F218Y;
T139P N167T, F168Y, S174C, D179E, A182C, A183R, K186R, A187R, F218Y;
N167T, F168Y, S174C, D179E, A182C, A183R, K186R, A187R, F218Y, H225S;
N167T, F168Y, S174C, D179E, A182C, A183R, K186R, A187R, F218Y L230R;
N167T, F168Y, S174C, D179E, A182C, A183R, K186R, A187R, F218Y, A245V;
N167T, F168Y, S174C, D179E, A182C, A183R, K186R, A187R, F218Y, H251E;
N167T, F168Y, S174C, D179E, A182C, A183R, K186R, A187R, F218Y, A281E;
N167T, F168Y, S174C, D179E, A182C, A183R, K186R, A187R, F218Y, L287T,
M289S;
N167T, F168Y, S174C, D179E, A182C, A183R, K186R, A187R, F218Y, S293S;
N167T, F168Y, S174C, D179E, A182C, A183R, K186R, A187R, F218Y, S304R;
D10N, F17Y, V28T, H39R, P57E, V58A, I59M, A63S, S65H, F69Y, A126S, T139P,
N167T, F168Y, S174C, D179E, A182C, A183R, K186R, A187R, F218Y, H225S,
L230R, A281E, L287T, M289S, F293S, S304R;

and/or
ii) is selected from the group consisting of: SEQ ID NOs: 2-35.
10. A method for obtaining a variant of a parent alpha/beta hydrolase, preferably having EC:3.1.1.-hydrolase activity, and/or increasing stability of said parent alpha/beta hydrolase, said method comprising: introducing into a parent alpha/beta hydrolase a substitution at one or more positions corresponding to positions 167, 168, 174, 218, 155, 175, 179, 182, 183, 186, 187, 268, 306, 10, 17, 28, 39, 47, 50, 57, 58, 59, 63, 65, 69, 126, 139, 225, 230, 245, 251, 281, 287, 289, 293 and 304 of SEQ ID NO: 1 (preferably using the numbering of SEQ ID NO: 1), wherein the variant has alpha/beta hydrolase activity; and recovering the variant (e.g., from a lysate, e.g., by the means of affinity purification); preferably said parent alpha/beta hydrolase is according to any one of the preceding items, further preferably said parent alpha/beta hydrolase having at least 95% sequence identity to the polypeptide of SEQ ID NO: 1.
11. The method according any one of the preceding items, wherein the variant has at least 71% (preferably: at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%), but less than 100% sequence identity to the polypeptide of SEQ ID NO: 1
12. A polynucleotide encoding the variant according to any one of the preceding items.
13. A nucleic acid construct comprising the polynucleotide according to any one of the preceding items.
14. An expression vector comprising the polynucleotide and/or nucleic acid construct according to any one of the preceding items.
15. A host cell comprising at least one of the following: i) the variant and/or the parent alpha/beta hydrolase according to any one of the preceding items; ii) the polynucleotide according to any one of the preceding items; iii) the nucleic acid construct according to any one of the preceding items; and/or iv) the expression vector according to any one of the preceding items.
16. The host cell according to any one of the preceding items, wherein said host cell is a recombinant host cell, preferably an isolated recombinant host cell.
17. The host cell according to any one of the preceding items, wherein the host cell is selected from a bacterial cell, yeast cell, mammalian cell, insect cell and plant cell, preferably from a bacterial cell and yeast cell.
18. A transgenic plant, transgenic seed or transgenic pollen grain comprising one or more (preferably a plurality) of the following:
   i) the variant and/or the parent alpha/beta hydrolase/s according to any one of the preceding items;
   ii) the polynucleotide according to any one of the preceding items;
   iii) the nucleic acid construct and/or expression vector according to any one of the preceding items; and/or
   iv) the recombinant host cell according to any one of the preceding items.
19. The transgenic plant, transgenic seed or transgenic pollen grain according to any one of the preceding items, capable of producing one or more variants according to any one of the preceding items, preferably said transgenic plant, transgenic seed or transgenic pollen grain is genetically-modified to produce one or more variants according to any one of the preceding items.
20. The transgenic plant, transgenic seed or transgenic pollen grain according to any one of the preceding items, wherein said transgenic plant, transgenic seed or transgenic pollen grain is homozygous for said (ii) and/or (iii).
21. A method for producing the variant according to any one of the preceding items, comprising: (i) cultivating the host cell according to any one of the preceding items under conditions suitable for expression of said variant; and (ii) recovering said variant.
22. A variant obtained by the method according any one of the preceding items.
23. Use of the variant and/or the parent alpha/beta hydrolase according any one of the preceding items, for/in/as one or more of the following:
   i) degradation of zearalenone or a derivative thereof;
   ii) food, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition;
   iii) manufacture/production of one or more of the following: foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition, and/or mixture/s thereof;
   iv) manufacture/production of silage, biogas, bioethanol, or sugar, preferably from sugar cane or sugar beets;
   v) prevention or treatment of mycotoxicosis.
24. A composition or kit comprising one or more of the following:
   (i) the variant and/or the parent alpha/beta hydrolase according any one of the preceding items, preferably said parent alpha/beta hydrolase having at least 95% sequence identity to the polypeptide of SEQ ID NO: 1;
   (ii) the polynucleotide according to any one of the preceding items;
   (iii) the nucleic acid construct according to any one of the preceding items;
   (iv) the expression vector according to any one of the preceding items; and/or
   (v) the host cell according to any one of the preceding items.
25. The composition or kit according any one of the preceding items, wherein said composition or kit is a veterinary composition, pharmaceutical composition or kit.
26. The composition or kit according any one of the preceding items, further comprising a veterinary composition, pharmaceutically acceptable carrier.
27. The composition or kit according any one of the preceding items, wherein the composition or kit is a food or feed additive or a food or feed product.
28. The composition or kit according to any one of the preceding items, further comprising a nutraceutically acceptable carrier and/or the parent alpha/beta hydrolase according any one of the preceding items, preferably said parent alpha/beta hydrolase having at least 95% sequence identity to the polypeptide of SEQ ID NO: 1.
29. The composition or kit according to any one of the preceding items, further comprising one or more of the following (e.g., as shown in Table 2 herein): (i) one or more further polypeptides capable of detoxifying (e.g., modifying and/or hydrolyzing) and/or binding Ochratoxin A (OTA) and/or at least one OTA derivative (e.g., ochratoxin B and/or ochratoxin C), preferably said further one or more polypeptides belonging to the M20 Peptidase aminoacylase 1-like protein 2-like amidohydrolase subfamily (e.g., M20 Peptidase ACY1L2 amidohydrolase subfamily, e.g., having identifier cd05672 according to the Conserved Domain Database (e.g., https://www.ncbi.nlm.nih.gov/Structure/cdd/cddsrv.cgi?uid=cd05672), and/or having aminopeptidase activity (e.g., EC 3.4.11.10), and/or comprise a carboxypeptidase activity (e.g., carboxypeptidase A and/or B activity, e.g., having EC 3.4.17.1 and/or EC 3.4.17.2 respectively) and/or thermolysin activity (e.g., having EC 3.4.24.27); (ii) one or more further polypeptides capable of detoxifying one or more mycotoxins (e.g., ZEN) and/or one or more plant- and/or bacteria-derived toxins (e.g., trichothecene mycotoxin/s such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin 1, B2, G1 or G2; fumonisins such as e.g. fumonisin 1, B2, B3 or B4; polypeptide mycotoxins such as e.g. beauvericin or enniatins; zearalenone; citrinin; patulin; ergot alkaloids such as e.g. ergotamine) and/or one or more plant- and/or bacteria-derived toxins (e.g. endotoxin, etc.), in particular said one or more further polypeptides capable of detoxifying one or more further mycotoxins and/or one or more plant- and/or bacteria-derived toxins, e.g., a fumonisin esterase (e.g. as disclosed in WO 2016/134387 A1) and/or a zearalenone lactonase (e.g. as disclosed in WO 2020/025580 A1) and/or an ergopeptine hydrolase (e.g. as disclosed in WO 2014/056006 A1); (iii) one or more organic absorbents (e.g., live/inactivated/lyophilized/dormant/dead whole-yeast or yeast-derived product such as e.g. yeast cell wall, or yeast oligosaccharides such as e.g. mannan) and/or inorganic absorbant (e.g., diatomaceous earth and/or clay mineral such as e.g. kaolins or kaolinites, smectites such as e.g. montmorillonites, illites or chlorites; in particular bentonite); (iv) one or more live, inactivated, lyophilized and/or dormant microorganisms capable of detoxifying one or more further mycotoxin (e.g., trichothecene mycotoxins such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin 1, B2, G1 or G2; fumonisins such as e.g. fumonisin 1, B2, B3 or B4; polypeptide mycotoxins such as e.g. beauvericin or enniatins; zearalenone; citrinin; patulin; ergot alkaloids such as e.g. ergotamine) and/or one or more plant- or bacterial-derived toxin (e.g. endotoxin), in particular said microorganism is a member of the Trichosporon or Apiotrichum genus (e.g. as disclosed in WO 03/053161 A1) or of the Coriobacteriaceae family (e.g. as disclosed in EP 3 501 526 A1); (v) one or more plant product (e.g., seaweed, preferably seaweed meal; and/or algae, preferably algae meal; and/or thistle, preferably thistle seeds; and/or glycyrrhiza plant preparation, preferably glycyrrhiza meal and/or glycyrrhiza extract e.g. as disclosed in WO 2018/121881 A1); (vi) one or more flavoring compound (e.g., plant extract e.g. from oregano, thyme, wintergreen, caraway, marjoram, mint, peppermint, anise, orange, lemon, fennel, star anise, clove, cinnamon and/or garlic; and/or essential oil such as e.g. D-limonene, γ-terpinene, p-cymene, 2-carene, linalool oxide, isomenthone, camphor, linalool, terpinen-4-ol, 2-isopropyl-1-methoxy-4-methylbenzene, L-menthol, ethylamine, α-terpineol, β-caryophyllene, D-carvone, methyl salicylate, α-caryophyllene, lavandulyl acetate, caryophyllene oxide, eugenol, thymol and/or carvacrol); (vii) one or more vitamin (e.g. vitamin A, D, E, K, C, 1, B2, B3, B4, B5, B6, B7, B8, B9, B12; in particular vitamin E).
30. The variant, parent alpha/beta hydrolase, polynucleotide, nucleic acid construct, expression vector, host cell, transgenic plant, transgenic seed, transgenic pollen grain, composition or kit according any one of the preceding items, for use as a medicament (e.g., for veterinary and/or pharmaceutical use, e.g., for animals and/or for humans) and/or in therapy, preferably for use in the prophylaxis or treatment of a disease, further preferably said parent alpha/beta hydrolase having at least 95% sequence identity to the polypeptide of SEQ ID NO: 1.
31. The variant, parent alpha/beta hydrolase, polynucleotide, nucleic acid construct, expression vector, host cell, transgenic plant, transgenic seed, transgenic pollen grain, composition or kit, for use in the prophylaxis or treatment of mycotoxicosis, preferably of hormonal disruption caused by zearalenone or a derivative thereof.
32. A method for prevention or treatment of mycotoxicosis, the method comprising: administering to a subject at risk or in need thereof one or more of the following: the variant, parent alpha/beta hydrolase, polynucleotide, nucleic acid construct, expression vector, host cell, transgenic plant, transgenic seed, transgenic pollen grain, composition or kit according any one of the preceding items.
33. The method or use according any one of the preceding items, wherein the variant, parent alpha/beta hydrolase, polynucleotide, nucleic acid construct, expression vector, host cell, transgenic plant, transgenic seed, transgenic pollen grain, composition or kit is administered in a therapeutically efficient amount.
34. A foodstuff, intermediate foodstuff; fodder, intermediate fodder; feed, intermediate feed; additive (preferably, foodstuff-, fodder- or feed additive), intermediate additive (preferably, foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition, and/or mixture/s thereof comprising one or more of the following: the variant, parent alpha/beta hydrolase, polynucleotide, nucleic acid construct, expression vector, host cell, transgenic plant, transgenic seed, transgenic pollen grain, composition or kit according to any one of the preceding items, preferably said parent alpha/beta hydrolase having at least 95% sequence identity to the polypeptide of SEQ ID NO: 1.
35. A method for degrading zearalenone or a derivative thereof, comprising:
   i) providing one or more variant/s according to any one of the preceding items and/or one or more parent alpha/beta hydrolase/s preferably according to any one of the proceeding items, preferably said parent alpha/beta hydrolase having at least 95% sequence identity to the polypeptide of SEQ ID NO: 1;
   ii) contacting one or more variant/s and/or parent/s with zearalenone or a derivative thereof (e.g., forming an enzyme-substrate mixture).
36. The method for degrading zearalenone or a derivative there according to any one of the preceding items, wherein the variant and/or the parent alpha/beta hydrolase has a specific activity of at least 0.6 μmol/min/mg (e.g., at least 0.7, at least 0.8, at least 0.9, at least 1.0 at least 1.1, at least 1.2, at least 1.3 or at least 1.4) at a substrate concentration of 0.5 ppm and at a pH of 5.0, preferably the variant has a specific activity of at least 1.35 μmol/min/mg (such as 1.39 or 1.57).

The invention is further illustrated by the following examples, however, without being limited to the example or by any specific embodiment of the examples.

### EXAMPLES OF THE INVENTION

### Example 1: Modification, Cloning, Expression of Polynucleotides Encoding Zearalenone-Cleaving Polypeptides and Purification of Zearalenone-Cleaving Polypeptides

Complete zearalenone-cleaving polypeptide genes (including a 6× histidine-tag fused at the C-terminal end of the polypeptides) were synthesized as nucleotide sequences, which were subsequently integrated in expression vectors by a public company (e.g. Twist Bioscience).

Chemically competent E. coli BL21 (DE3) were transformed with the expression vectors containing complete zearalenone-cleaving polypeptide genes. Transformants were selected via antibiotic resistance of the expression vector. Any other suitable host cell may also be used for this task.

For the preparation of chemically competent E. coli BL21 (DE3), cells were inoculated in LB media (e.g. Luria/Miller, granulated, 6673, Carl Roth GmbH+CoKG, 25.0 g/L) and grown under constant shaking over night at 37.0° C. The overnight culture was inoculated for the main culture in LB media and grown under constant shaking at 37.0° C. to OD₆₀₀ of 0.65. 15.0 minutes prior to harvest, the main culture was cooled to 4.0° C. and for all further steps the cells were kept at 4.0° C. whenever possible or stated otherwise. The cells were harvested at 2000.0× g for 10.0 minutes at 4.0° C. The cell pellet was resuspended in 5.0 ml TSS buffer (LB media, 10.0% polyethyleneglycol-6000, 5.0% dimethylsulfoxide, 20.0 mM magnesium chloride) per 100.0 ml main culture.

For the transformation of chemically competent E. coli BL21 (DE3), the competent cell solution was diluted 1:1.25 with 5×KCM buffer (500.0 mM potassium chloride, 150.0 mM calcium chloride, 250.0 mM magnesium chloride). 50.0 μl of the prepared cell solution was supplied with 5.0 μl of the expression vectors containing complete zearalenone-cleaving polypeptide genes and incubated for 10.0 minutes at 4.0° C. A heat-shock was performed at 42.0° C. for 1.5 minutes, followed by incubation for 1.0 minute at 4.0° C. The cells were regenerated in 1.0 ml LB media for 1.5 hours at 37.0° C. 100.0 μl of the cells were inoculated in 900.0 μl LB media with antibiotic (e.g. kanamycin 50.0 μg/ml) and cultivated under constant shaking over night at 37.0° C.

Complete zearalenone-cleaving polypeptide genes were intracellularly expressed from positive transformants using commercially available auto-induction media.

500.0 μl of the overnight culture were inoculated in 9.5 ml auto-induction media (e.g. Novagen Overnight Express™ Instant TB Medium, 71491, Sigma-Aldrich Handels GmbH) including antibiotic, (e.g. kanamycin 50.0 μg/ml) in a 50.0 ml reaction tube and covered with sealing membrane for a high rate of gas exchange (e.g. Breathe.Easier sealing membrane, Z763624, Sigma-Aldrich Handels GmbH). The expression culture was incubated under constant shaking for 24.0 hours at 37.0° C. The cells were harvested at 2000.0× g for 10.0 minutes at room temperature.

Harvested cells from the expression step were lysed via sonication. Zearalenone-cleaving polypeptides were obtained from cleared lysates by 6× Histidine-tag affinity purification. Molecular weight and purity were confirmed via SDS-PAGE analysis. Concentration of zearalenone-cleaving polypeptides was determined via NanoDrop™ analysis.

Harvested cells were resuspended in 2.0 ml binding buffer (20.0 mM sodium phosphate, 500.0 mM sodium chloride, 60.0 mM imidazole, pH 7.4) at 4.0° C. The suspension was treated on a sonicator until complete cell lysis at 4.0° C. (e.g. Q700-220+Q4579, Amplitude 50.0—processing time 8.0 minutes—pulse 20.0 seconds—pause 40.0 seconds, QSonica L.L.C).

The lysates were cleared by centrifugation at 21000×g for 15.0 minutes at 4.0° C. 1.4 ml of the supernatant were purified on HIS-Spin trap columns according to manufacturer protocol (e.g. HIS-Spin Trap, 28-4013-53, GE Healthcare GmbH). The zearalenone-cleaving polypeptides were eluted in 400.0 μl elution buffer (20.0 mM sodium phosphate, 500.0 mM sodium chloride, 500.0 mM imidazole, pH 7.4), and this preparations were used for enzymatic characterization.

To confirm molecular weight and purity of the purified zearalenone-cleaving polypeptides, dilutions of the elutes were supplied with loading dye (e.g. 2× loading dye: 0.125 M tris(hydroxymethyl)aminomethan/hydrochloric acid pH 6.8, 20.0% glycerol, 4.0% sodiumdodecylsulphate, 0.02% bromphenol blue, 2% beta-mercaptoethanol), boiled and separated on a 12.0% polyacrylamid gel (e.g. Mini-PROTEAN® Tetra Cell System, 1658004EDU+12% Mini-PROTEAN® TGX™ Precast Protein Gels, 4561043EDU, Bio-Rad Laboratories GmbH) (10× running buffer: 0.125 M tris(hydroxymethyl)aminomethane, 1.25 M glycine, 0.5% sodiumdodecylsulphate, 200 V, 40 minutes). The peptide bands were visualized by Coomassie staining and compared to a standard peptide marker.

To determine the concentration of the purified zearalenone-cleaving polypeptides, absorption at 280.0 nm was determined (e.g. NanoDrop™ One, ND-ONE-W, Thermo Fisher Scientific Inc.) and concentration was calculated according to Lambert-Beer law.

Analysis of the Degree of Enzyme Oligomerisation (Blue-Native-PAGE—BN-PAGE)

Comparison of the native molecular weight (oligomeric conformation) between single purified zearalenone-cleaving polypeptides was done via Blue-Native-PAGE. Dilutions of the elutes were supplied with not denaturating loading dye (e.g. 2× loading dye: 0.126 M tris(hydroxymethyl)aminomethan/hydrochloric acid pH 6.8, 20.0% glycerol, 4.0% Coomassie Blue G-250) and separated on a 4-15% polyacrylamid gel under native conditions (e.g. Mini-PROTEAN® Tetra Cell System, 1658004EDU+4-15% Mini-PROTEAN® TGX™ Precast Protein Gels, 4561083EDU, Bio-Rad Laboratories GmbH) (5× Anode Buffer: 0.125 M Tris(hydroxymethyl)aminomethane, 0.960 M Glycine, 1× Kathode Buffer: Anode Buffer+0.02% Coomassie Blue G250; 200 V, 40 minutes). The peptide bands were visualized by Coomassie staining. The oligomeric state of selected zearalenone-cleaving polypeptides was determined by size exclusion chromatography, SEC-MALS or X-Ray crystallography. Homo-dimers (e.g. SEQ ID NO. 1) migrated faster across the BN-PAGE gel matrix, while homo-tetramers (e.g. SEQ ID NO. 2-35) were retained in the gel matrix to a higher extent. The oligomeric conformation of the zearalenone-cleaving polypeptides was determined relatively to the migration of homo-dimeric and homo-tetrameric peptides.

Determination of the melting point (Tm) of zearalenone-cleaving polypeptides: The melting point of the purified zearalenone-cleaving polypeptides was determined with a thermal shift assay, based on the fluorescence of the dye SYPRO Orange during protein denaturation. While water quenches its fluorescence, binding to unfolded hydrophobic surfaces on the denaturated protein results in an increase in fluorescence.

Purified zearalenone-cleaving polypeptides were diluted in Teorell Stenhagen Buffer at pH 7.5 (Ostling and Virtama, Acta Physiologica Scandinavica, 1946, 11:4, 289-293) to 0.2 mg/ml. Sypro Orange 5000× stock (Sypro Orange, S5692, Sigma-Aldrich Handels GmbH) was diluted 1:100 in Teorell Stenhagen Buffer pH 7.5. 15.0 μl of the diluted zearalenone-cleaving polypeptides were supplied with 3.0 μl of the diluted Sypro Orange solution and 12.0 μl Teorell Stenhagen Buffer pH 7.5 in a 96-well PCR-plate which was sealed with a transparent heat sealing film suitable for fluorescence measurements. Melting curves were determined on a qPCR-cycler with linear increasing temperature from 20.0 to 95.0° C. in 20.0 minutes and 147 measurement points per measurement curve (extinction at 460.0 nm, emission at 550.0 nm) (e.g. Mastercycler qPCR ep realplex 2s, 6300000604, Eppendorf AG).

The melting point was determined at the point of fluorescence signal inflection (in other words, at the temperature with the highest rate of fluorescence signal increase). The fluorescence intensity of the 147 measurement points was plotted against the temperature gradient. The differential for each of the 147 measurement points was calculated according to dFluorescencesignal/dTemperature for two subsequent measurement points. The temperature at which the highest value of dF/dT was calculated is the maximum of first derivative, which represents the inflection point and thus the melting point of the zearalenone-cleaving polypeptides.

TABLE 3
Melting point (Tm) of zearalenone-cleaving polypeptides.

| SEQ | | oligomeric | Tm |
|---|---|---|---|
| ID NO. | amino acid substitutions compared to SEQ ID NO: 1 | conformation | (° C.) |
| 1 | — | dimer | 50.0 |
| 2 | N167T, F168Y, S174C, F218Y | tetramer | 54.0 |
| 3 | N167T | tetramer | 53.1 |
| 4 | N167S | tetramer | 53.0 |
| 5 | N167A | tetramer | 52.2 |
| 6 | N167C | tetramer | 52.9 |
| 7 | N167L | tetramer | 52.3 |
| 8 | N167T, S174T | tetramer | 53.5 |
| 9 | N167T, S174C | tetramer | 53.8 |
| 10 | R155K, N167T, S174C, V175A | tetramer | 53.9 |
| 11 | R155K, N167T, S174C, V175T | tetramer | 53.8 |
| 12 | N167S, S174C | tetramer | 54.2 |
| 13 | N167T, S174C, F218Y | tetramer | 54.3 |
| 14 | N167T, F168Y, S174C | tetramer | 54.3 |
| 15 | N167T, F168Y, S174C, D179E, A182C, A183R, | tetramer | 65.2 |
| | K186R, A187R, F218Y | | |
| 16 | N167T, F168Y, S174C, D179E, A182C, A183R, | tetramer | 63.2 |
| | K186R, A187R, F218Y, Q268L, V306I | | |
| 17 | D10N, N167T, F168Y, S174C, D179E, A182C, | tetramer | 64.2 |
| | A183R, K186R, A187R, F218Y | | |
| 18 | F17Y, I59M, N167T, F168Y, S174C, D179E, A182C, | tetramer | 64.2 |
| | A183R, K186R, A187R, F218Y | | |
| 19 | V28T, N167T, F168Y, S174C, D179E, A182C, | tetramer | 65.8 |
| | A183R, K186R, A187R, F218Y | | |
| 20 | H39R, N167T, F168Y, S174C, D179E, A182C, | tetramer | 64.7 |
| | A183R, K186R, A187R, F218Y | | |
| 21 | E47S, G50E, N167T, F168Y, S174C, D179E, | tetramer | 59.6 |
| | A182C, A183R, K186R, A187R, F218Y | | |
| 22 | P57E, V58A, I59M, N167T, F168Y, S174C, D179E, | tetramer | 63.2 |
| | A182C, A183R, K186R, A187R, F218Y | | |
| 23 | A63S, S65H, N167T, F168Y, S174C, D179E, | tetramer | 65.3 |
| | A182C, A183R, K186R, A187R, F218Y | | |
| 24 | F69Y, N167T, F168Y, S174C, D179E, A182C, | tetramer | 64.2 |
| | A183R, K186R, A187R, F218Y | | |
| 25 | A126S, N167T, F168Y, S174C, D179E, A182C, | tetramer | 64.7 |
| | A183R, K186R, A187R, F218Y | | |
| 26 | T139P N167T, F168Y, S174C, D179E, A182C, | tetramer | 67.8 |
| | A183R, K186R, A187R, F218Y | | |
| 27 | N167T, F168Y, S174C, D179E, A182C, A183R, | tetramer | 64.2 |
| | K186R, A187R, F218Y, H225S | | |
| 28 | N167T, F168Y, S174C, D179E, A182C, A183R, | tetramer | 69.8 |
| | K186R, A187R, F218Y L230R | | |
| 29 | N167T, F168Y, S174C, D179E, A182C, A183R, | tetramer | 62.7 |
| | K186R, A187R, F218Y, A245V | | |
| 30 | N167T, F168Y, S174C, D179E, A182C, A183R, | tetramer | 66.8 |
| | K186R, A187R, F218Y, H251E | | |
| 31 | N167T, F168Y, S174C, D179E, A182C, A183R, | tetramer | 65.3 |
| | K186R, A187R, F218Y, A281E | | |
| 32 | N167T, F168Y, S174C, D179E, A182C, A183R, | tetramer | 64.7 |
| | K186R, A187R, F218Y, L287T, M289S | | |
| 33 | N167T, F168Y, S174C, D179E, A182C, A183R, | tetramer | 63.2 |
| | K186R, A187R, F218Y, S293S | | |
| 34 | N167T, F168Y, S174C, D179E, A182C, A183R, | tetramer | 64.7 |
| | K186R, A187R, F218Y, S304R | | |
| 35 | D10N, F17Y, V28T, H39R, P57E, V58A, I59M, | tetramer | 65.8 |
| | A63S, S65H, F69Y, A126S, T139P, N167T, F168Y, | | |
| | S174C, D179E, A182C, A183R, K186R, A187R, | | |
| | F218Y, H225S, L230R, A281E, L287T, M289S, | | |
| | F293S, S304R | | |

Determination of the Specific Activity

A zearalenone degradation assay buffer (118.5 mM NaCl, 8.55 mM acetic acid, 14.9 mM acetate, 0.1 mg/ml BSA, pH 5.0, (Jantratid, E., Janssen, N., Reppas, C. & Dressman, J. B. (2008): Dissolution media simulating conditions in the proximal human gastrointestinal tract: An update. Pharmaceutical Research 25 (7): 1663-1576), containing 0.1 mg/ml bovine serum albumin and 0.52 ppm zearalenone as substrate) was prepared, prewarmed at 37.0° C. and 960 μl aliquots of assay buffer were transferred into the reaction tubes of a 96 deep well plate. The plate was sealed with a movable lid and kept at 37.0° C. in a DWP-thermoshaker until the start of the zearalenone degradation assay.

Enzyme working solutions were prepared with the purified zearalenone-cleaving polypeptides by dilution in sample buffer (Teorell Stenhagen buffer at pH 7.5, containing 0.1 mg/mi bovine serum albumin) to a 25.0-fold higher concentration as analyzed in the final zearalenone degradation assay, with a concentration of zearalenone-cleaving polypeptides to efficiently degrade zearalenone under the given conditions.

To start the zearalenone degradation assay, 40.0 μl of the enzyme working solutions were added to the tubes containing the 960.0 μl assay buffer, hereby achieving a final ZEN concentration of 0.5 ppm in the assay reaction. The addition of the enzyme working solution with pH 7.5 did not change the pH 5.0 of the zearalenone degradation reactions.

The zearalenone degradation reactions were incubated in the DWP-thermoshaker under constant shaking at 37.0° C. Immediately after the zearalenone degradation reaction was started, it was mixed by re-suspending with the pipet tip and a 0.0 h sample of 120.0 μl was transferred into a tube of a new PCR plate and sealed with a cap. Additional samples were drawn from the zearalenone degradation reaction after several time points (e.g. 5.0, 10.0, 20.0, 30.0, 45.0, 60.0, 90.0 minutes). As soon as a sample was drawn from the degradation reaction, the zearalenone-cleaving polypeptide in this sample was heat-inactivated by incubation for 10.0 minutes at 99.0° C. in a thermo-block (e.g. Thermal Shake lite, 460-029, VWR). Subsequently, the tube was centrifuged (3.0 minutes, room temperature, 2500× g) and 90.0 μl of the supernatant was transferred into a skirted PCR plate (e.g. twin.tec® PCR plate, 0030128648, Eppendorf AG), which was closed with a heat sealing film (e.g. Heat Sealing Film, 0030127838, Eppendof AG). These sample plates were stored at 4.0° C. until HPLC-FLD measurement. Zearalenone concentration was determined by a modified HPLC-FLD method as described in Vekiru et al. (Vekiru et al. (2016) ‘Isolation and characterization of enzymatic zearalenone hydrolysis reaction products’ World Mycotoxin Journal 9:353-363).

Analysis was performed on an HPLC-FLD operated at an extinction of 278.0 nm and an emission at 465.0 nm. The retention time of zearalenone was 1.8 min when separation as done on a Kinetex 5 μm EVO C18 100 A 50.0×2.1 mm column (OOB-4633-AN, Phenomenex Inc.) at 40.0° C. by using solvent A: 95.0% acetonitrile+4.9% water+0.1% formic acid and solvent B: 5.0% acetonitrile+94.9% water+0.1% formic acid when using a gradient: 0.0-0.5 minutes 10% phase B, 0.5-2.0 minutes linear increase to 50.0% phase B, then reduced to 10% phase B in 0.1 minutes, which was continued for the total run time of 2.3 minutes. The flow rate was set at 1.5 ml/min and the injection volume to 5.0 μl. A similar acquisition setup was performed for various analysis.

Quantification of zearalenone was based on calibration with external standards of zearalenone. The zearalenone concentration (μM) of the taken reaction samples were plotted against the sampling time point. A slope of the linear zearalenone degradation over time was calculated in μmol zearalenone reduction per minute in the enzymatic reaction volume. By considering possible dilutions of the purified zearalenone-cleaving polypeptides and by including these appropriate dilution factors in the calculation, the specific enzymatic activity determined in μmol zearalenone degradation per minute per mg purified enzyme (μmol ZEN/min/mg) was calculated.

TABLE 4
Specific enzyme activities at pH 5.0.

| | specific activity |
|---|---|
| Enzyme Identifikation | (μmol ZEN/min/mg) |
| prior art comparative sequence 1 | 0.00 |
| prior art comparative sequence 2 | 0.00 |
| SEQ ID NO. 1 of the present invention | 1.57 |
| SEQ ID NO. 28 of the present invention | 1.39 |

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes.

Other embodiments are within the following claims. In addition, where features or aspects of the invention are described in terms of Markush groups, those skilled in the art will recognize that the invention is also thereby described in terms of any individual member or subgroup of members of the Markush group.

1. A variant of a parent alpha/beta hydrolase, the variant comprising a substitution at one or more positions corresponding to positions: 167, 168, 174, 218, 155, 175, 179, 182, 183, 186, 187, 268, 306, 10, 17, 28, 39, 47, 50, 57, 58, 59, 63, 65, 69, 126, 139, 225, 230, 245, 251, 281, 287, 289, 293 and 304 of SEQ ID NO: 1 (preferably using the numbering of SEQ ID NO: 1), wherein said variant has alpha/beta hydrolase activity and wherein the variant is a polypeptide having at least 71% (preferably: at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%), but less than 100% sequence identity to the polypeptide of SEQ ID NO: 1, preferably said variant having a hydrolase EC: 3.1.1.-activity.

2. The variant according to any one of the preceding claims, wherein said variant having one or more of the following:
i) an improved property relative to the parent alpha/beta hydrolase, wherein said improved property comprises an increased temperature stability, preferably as compared to SEQ ID NO: 1; is capable of tetramerization, preferably having a tetrameric quaternary structure, further preferably said tetramerization is a homo-tetramerization;
ii) a melting point (Tm) greater than 50° C., preferably said Tm is in the range from about 51° C. to about 70° C.;
iii) capable of degrading zearalenone and/or derivatives thereof;
iv) comprising at least one (preferably at least two, more preferably at least three, most preferably at least four) amino acid motif/s having a sequence identity of at least 80% (preferably at least 83%, more preferably at least 90%) to an amino acid sequence selected from:
a) SEQ ID NO: 36 (QVDLGEX₁X₂MN), wherein X, is V or I, preferably V, and wherein X₂ is V or T, preferably V;
b) SEQ ID NO: 37 (EYDPEWX₃RX₄FX₅EGTV), wherein X₃ is G or A, preferably A, and wherein X₄ is A or V, preferably A, and wherein X₅ is F or Y;
c) SEQ ID NO: 38 (MLX₆QVKTPX₇LX₃THH), wherein X₆ is S or G, preferably S, and wherein X₇ is I or V, preferably 1, and wherein X₃ is I or L, preferably I; and
d) SEQ ID NO: 39 (PAX₉LLX₁₀PEQTGSWWSYEX₁₁X₁₂X₁₃GLLX₁₄EX₁₅FHVX₁₆AVDX₁₇RG QGRSX₁₈WTPX₁₉RYSLDNFGNDLVRFIX₂₀LVX₂₁KRPVX₂₂VX₂₃GNSSG GX₂₄LAAWLSAYX₂₅MPGQX₂₆RX₂₇X₂₈LCEDX₂₉X₃₀FFASELVPAX₃₁GHS VX₃₂QX₃₃AGPX₃₄FELX₃₅R), wherein X₉ is V or L, preferably L, and wherein X₁₀ is L or I, preferably L, and wherein X₁₁ is P or E, preferably P, and wherein X₁₂ is V or A, preferably V, and wherein X₁₃ is I or M, preferably I, and wherein X₁₄ is A or S, preferably A, and wherein X₁₅ is S, N or H, preferably S, and wherein X₁₆ is F or Y, preferably F, and wherein X₁₇ is I or L, preferably I, and wherein X₁₈ is T or S, preferably T, and wherein X₁₉ is K or R, preferably K, and wherein X₂₀ is S, A or N, preferably S, and wherein X₂₁ is V or I, preferably V, and wherein X₂₂ is I or V, preferably I, and wherein X₂₃ is S or A, preferably S, and wherein X₂₄ is V or L, preferably V, and wherein X₂₅ is A or S, preferably A, and wherein X₂₆ is I or L, preferably I, and wherein X₂₇ is A or G, preferably A, and wherein X₂₈ is A or V, preferably A, and wherein X₂₉ is T, A or P, preferably T, and wherein X₃₀ is P or A, preferably P, and wherein X₃₁ is Y or H, preferably H, and wherein X₃₂ is L or R, preferably R, and wherein X₃₃ is A or G, preferably A, and wherein X₃₄ is A or V, preferably A, and wherein X₃₅ is Y or F, preferably Y.

3. The variant according to any one of the preceding claims, wherein said variant comprises one or more of the following substitutions:
X167T, X168Y, X174C, X218Y;
X167T;
X167S;
X167A;
X167C;
X167L;
X167T, X174T;
X167T, X174C;
X155K, X167T, X174C, X175A;
X155K, X167T, X174C, X175T;
X167S, X174C;
X167T, X174C, X218Y;
X167T, X168Y, X174C;
X167T, X168Y, X174C, X179E, X182C, X183R, X186R, X187R, X218Y;
X167T, X168Y, X174C, X179E, X182C, X183R, X186R, X187R, X218Y, X268L,
X306I;
X10N, X167T, X168Y, X174C, X179E, X182C, X183R, X186R, X187R, X218Y;
X17Y, X59M, X167T, X168Y, X174C, X179E, X182C, X183R, X186R, X187R,
X218Y;
X28T, X167T, X168Y, X174C, X179E, X182C, X183R, X186R, X187R, X218Y;
X39R, X167T, X168Y, X174C, X179E, X182C, X183R, X186R, X187R, X218Y;
X47S, X50E, X167T, X168Y, X174C, X179E, X182C, X183R, X186R, X187R,
X218Y;
X57E, X58A, X59M, X167T, X168Y, X174C, X179E, X182C, X183R, X186R, X187R,
X218Y;
X63S, X65H, X167T, X168Y, X174C, X179E, X182C, X183R, X186R, X187R,
X218Y;
X69Y, X167T, X168Y, X174C, X179E, X182C, X183R, X186R, X187R, X218Y;
X126S, X167T, X168Y, X174C, X179E, X182C, X183R, X186R, X187R, X218Y;
X139P X167T, X168Y, X174C, X179E, X182C, X183R, X186R, X187R, X218Y;
X167T, X168Y, X174C, X179E, X182C, X183R, X186R, X187R, X218Y, X225S;
X167T, X168Y, X174C, X179E, X182C, X183R, X186R, X187R, X218Y X230R;
X167T, X168Y, X174C, X179E, X182C, X183R, X186R, X187R, X218Y, X245V;
X167T, X168Y, X174C, X179E, X182C, X183R, X186R, X187R, X218Y, X251E;
X167T, X168Y, X174C, X179E, X182C, X183R, X186R, X187R, X218Y, X281E;
X167T, X168Y, X174C, X179E, X182C, X183R, X186R, X187R, X218Y, X287T,
X289S;
X167T, X168Y, X174C, X179E, X182C, X183R, X186R, X187R, X218Y, X293S;
X167T, X168Y, X174C, X179E, X182C, X183R, X186R, X187R, X218Y, X304R;
X10N, X17Y, X28T, X39R, X57E, X58A, X59M, X63S, X65H, X69Y, X126S, X139P,
X167T, X168Y, X174C, X179E, X182C, X183R, X186R, X187R, X218Y, X225S,
X230R, X281E, X287T, X289S, X293S, X304R;
and/or
X155R, X167T, X168Y, X174C, X175V, X179E, X182C, X183R, X186R, X187R,
X189H, X190S, X192V, X202V, X216V, X218Y, X287T, X289S;
X155R, X167T, X168Y, X174C, X175V, X218Y;
X155R, X167T, X168Y, X174C, X175V;
X155R, X167T, X174C, X175V, X218Y;
X155R, X167T, X168Y, X174C, X175V, X179E, X182C, X183R, X186R, X187R,
X218Y;
X155R, X167T, X168Y, X174C, X175V, X189H, X190S, X192V, X218Y;
X155R, X167T, X168Y, X174C, X175V, X179E, X182C, X183R, X186R, X187R,
X189H, X190S, X192V, X218Y;
X155R, X167T, X168Y, X174C, X175V, X179E, X182C, X183R, X186R, X187R,
X202V, X218Y;
X155R, X167T, X168Y, X174C, X175V, X179E, X182C, X183R, X186R, X187R,
X202V, X218Y, X268L, X306I;
X34V, X100N, X140A, X155R, X167T, X168Y, X174C, X175V, X179E, X182C,
X183R, X186R, X187R, X202V, X218Y, X225N, X266R, X268L, X269Q, X306I;
X155R, X167T, X168Y, X174C, X175V, X179E, X182C, X183R, X186R, X187R,
X218Y, X287S, X289S;
X155R, X167T, X168Y, X174C, X175V, X179E, X182C, X183R, X186R, X187R,
X202V, X218Y, X287S, X289S;
X155R, X167T, X168Y, X174C, X175V, X179E, X182C, X183R, X186R, X187R,
X202V, X218Y, X268L, X287S, X289S, X306I;
X34V, X100N, X140A, X155R, X167T, X168Y, X174C, X175V, X179E, X182C,
X183R, X186R, X187R, X202V, X218Y, X225N, X266R, X268L, X269Q, X287S,
X289S, X306I;
or an equivalent amino acid substitutions thereof.

4. The variant according to any one of the preceding claims, wherein said parent alpha/beta hydrolase, preferably having EC:3.1.1.-hydrolase activity, is selected from the group consisting of:
i) a polypeptide having at least 60% (preferably at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, or at least 99.5%, or 100%) sequence identity to the polypeptide of SEQ ID NO: 1;
ii) a fragment of the polypeptide of SEQ ID NO: 1, wherein said fragment has alpha/beta hydrolase activity, preferably an EC:3.1.1.-hydrolase activity.

5. The variant according to any one of preceding claims, wherein said variant:
i) comprises or consists of a substitution/s selected from the group consisting of:
N167T, F168Y, S174C, F218Y;
N167T;
N167S;
N167A;
N167C;
N167L;
N167T, S174T;
N167T, S174C;
R155K, N167T, S174C, V175A;
R155K, N167T, S174C, V175T;
N167S, S174C;
N167T, S174C, F218Y;
N167T, F168Y, S174C;
N167T, F168Y, S174C, D179E, A182C, A183R, K186R, A187R, F218Y;
N167T, F168Y, S174C, D179E, A182C, A183R, K186R, A187R, F218Y, Q268L,
V306I;
D10N, N167T, F168Y, S174C, D179E, A182C, A183R, K186R, A187R, F218Y;
F17Y, I59M, N167T, F168Y, S174C, D179E, A182C, A183R, K186R, A187R,
F218Y;
V28T, N167T, F168Y, S174C, D179E, A182C, A183R, K186R, A187R, F218Y;
H39R, N167T, F168Y, S174C, D179E, A182C, A183R, K186R, A187R, F218Y;
E47S, G50E, N167T, F168Y, S174C, D179E, A182C, A183R, K186R, A187R,
F218Y;
P57E, V58A, I59M, N167T, F168Y, S174C, D179E, A182C, A183R, K186R, A187R,
F218Y;
A63S, S65H, N167T, F168Y, S174C, D179E, A182C, A183R, K186R, A187R,
F218Y;
F69Y, N167T, F168Y, S174C, D179E, A182C, A183R, K186R, A187R, F218Y;
A126S, N167T, F168Y, S174C, D179E, A182C, A183R, K186R, A187R, F218Y;
T139P N167T, F168Y, S174C, D179E, A182C, A183R, K186R, A187R, F218Y;
N167T, F168Y, S174C, D179E, A182C, A183R, K186R, A187R, F218Y, H225S;
N167T, F168Y, S174C, D179E, A182C, A183R, K186R, A187R, F218Y L230R;
N167T, F168Y, S174C, D179E, A182C, A183R, K186R, A187R, F218Y, A245V;
N167T, F168Y, S174C, D179E, A182C, A183R, K186R, A187R, F218Y, H251E;
N167T, F168Y, S174C, D179E, A182C, A183R, K186R, A187R, F218Y, A281E;
N167T, F168Y, S174C, D179E, A182C, A183R, K186R, A187R, F218Y, L287T,
M289S;
N167T, F168Y, S174C, D179E, A182C, A183R, K186R, A187R, F218Y, S293S;
N167T, F168Y, S174C, D179E, A182C, A183R, K186R, A187R, F218Y, S304R;
D10N, F17Y, V28T, H39R, P57E, V58A, I59M, A63S, S65H, F69Y, A126S, T139P,
N167T, F168Y, S174C, D179E, A182C, A183R, K186R, A187R, F218Y, H225S,
L230R, A281E, L287T, M289S, F293S, S304R;
and/or
ii) is selected from the group consisting of: SEQ ID NOs: 2-35.

6. A method for obtaining a variant of a parent alpha/beta hydrolase, preferably having EC:3.1.1.-hydrolase activity, and/or increasing stability of said parent alpha/beta hydrolase, said method comprising: introducing into said parent alpha/beta hydrolase a substitution at one or more positions corresponding to positions 167, 168, 174, 218, 155, 175, 179, 182, 183, 186, 187, 268, 306, 10, 17, 28, 39, 47, 50, 57, 58, 59, 63, 65, 69, 126, 139, 225, 230, 245, 251, 281, 287, 289, 293 and 304 of SEQ ID NO: 1 (preferably using the numbering of SEQ ID NO: 1), wherein the variant has alpha/beta hydrolase activity; and recovering the variant; preferably said parent alpha/beta hydrolase is according to any one of the preceding claims, further preferably said parent alpha/beta hydrolase having at least 95% sequence identity to the polypeptide of SEQ ID NO: 1.

7. A polynucleotide encoding the variant according to any one of the preceding claims.

8. A nucleic acid construct comprising the polynucleotide according to any one of the preceding claims

9. An expression vector comprising the polynucleotide and/or nucleic acid construct according to any one of the preceding claims.

10. A recombinant host cell comprising at least one of the following:
i) variant and/or the parent alpha/beta hydrolase according to any one of the preceding claims;
ii) polynucleotide according to any one of the preceding claims;
iii) nucleic acid construct according to any one of the preceding claims; and/or
iv) expression vector according to any one of the preceding claims.

11. A transgenic plant, transgenic seed or transgenic pollen grain comprising one or more (preferably a plurality) of the following:
i) variant and/or parent alpha/beta hydrolase/s according to any one of the preceding claims;
ii) polynucleotide according to any one of the preceding claims;
iii) nucleic acid constructs according to any one of the preceding claims;
iv) expression vector according to any one of the preceding claims; and/or
v) recombinant host cell according to any one of the preceding claims.

12. A foodstuff, intermediate foodstuff; fodder, intermediate fodder; feed, intermediate feed; additive (preferably, foodstuff-, fodder- or feed additive), intermediate additive (preferably, foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition, and/or mixture/s thereof comprising one or more of the following:
i) variant and/or parent alpha/beta hydrolase according to any one of the preceding claims; preferably said parent alpha/beta hydrolase having at least 95% sequence identity to the polypeptide of SEQ ID NO: 1;
ii) polynucleotide according to any one of the preceding claims;
iii) nucleic acid construct according to any one of the preceding claims;
iv) expression vector according to any one of the preceding claims;
v) recombinant host cell according to any one of the preceding claims;
vi) transgenic plant, transgenic seed and/or transgenic pollen grain according to any one of the preceding claims.

13. A composition or kit comprising one or more of the following:
(i) variant and/or the parent alpha/beta hydrolase according any one of the preceding claims, preferably said parent alpha/beta hydrolase having at least 95% sequence identity to the polypeptide of SEQ ID NO: 1;
(ii) polynucleotide according to any one of the preceding claims;
(iii) nucleic acid construct according to any one of the preceding claims;
(iv) expression vector according to any one of the preceding claims;
(v) recombinant host cell according to any one of the preceding claims;
(vi) transgenic plant, transgenic seed or transgenic pollen grain according to any one of the preceding claims; and/or
(vii) foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote;
veterinary composition; pharmaceutical composition and/or mixture/s thereof according to any one of the preceding claims.

14. The composition or kit according to any one of the preceding claims, further comprising a nutraceutically acceptable carrier and/or parent alpha/beta hydrolase according any one of the preceding claims, preferably said parent alpha/beta hydrolase having at least 95% sequence identity to the polypeptide of SEQ ID NO: 1.

15. The variant, parent alpha/beta hydrolase, polynucleotide, nucleic acid construct, expression vector, recombinant host cell, transgenic plant, transgenic seed, transgenic pollen grain, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof, composition and/or kit according any one of the preceding claims, for use as a medicament and/or in therapy, preferably for use in the prophylaxis or treatment of a disease, further preferably said parent alpha/beta hydrolase having at least 95% sequence identity to the polypeptide of SEQ ID NO: 1.

16. The variant, parent alpha/beta hydrolase, polynucleotide, nucleic acid construct, expression vector, recombinant host cell, transgenic plant, transgenic seed, transgenic pollen grain, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof, composition and/or kit according any one of the preceding claims, for use in the prophylaxis or treatment of mycotoxicosis, preferably of hormonal disruption caused by zearalenone or a derivative thereof.

17. Use of the variant, parent alpha/beta hydrolase polynucleotide, nucleic acid construct, expression vector, recombinant host cell, transgenic plant, transgenic seed, transgenic pollen grain, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof according any one of the preceding claims, for one or more of the following:
i) degradation of zearalenone and/or a derivative thereof;
ii) food, foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition;
iii) manufacture/production of one or more of the following: foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition, and/or mixture/s thereof;
iv) manufacture/production of silage, biogas, bioethanol, or sugar, preferably from sugar cane or sugar beets;
v) prevention or treatment of mycotoxicosis.

18. A method for degrading zearalenone and/or a derivative thereof, comprising:
a) providing one or more of the following:
i) variant and/or the parent alpha/beta hydrolase according any one of the preceding claims, preferably said parent alpha/beta hydrolase having at least 95% sequence identity to the polypeptide of SEQ ID NO: 1;
ii) recombinant host cell according to any one of the preceding claims;
iii) transgenic plant according to any one of the preceding claims;
iv) foodstuff, intermediate foodstuff; fodder, intermediate fodder; feed, intermediate feed; additive (preferably foodstuff-, fodder- or feed additive), intermediate additive (preferably foodstuff-, fodder- or feed intermediate additive); detoxifying agent, intermediate detoxifying agent; nutritional supplement, intermediate nutritional supplement; prebiotic, intermediate prebiotic, silage inoculant; antidote; veterinary composition; pharmaceutical composition and/or mixture/s thereof according to any one of the preceding claims; and/or
v) composition or kit according to any one of the preceding claims;
b) contacting one or more from (a) with zearalenone and/or a derivative thereof.


SEQUENCE LISTING (39 sequences)

SEQ ID NO: 1 - protein, 316 aa, Aeromicrobium sp.
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRNFLGDQWSVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 2 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWDGFAAAAKASPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 3 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTFLGDQWSVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 4 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRSFLGDQWSVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 5 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRAFLGDQWSVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 6 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRCFLGDQWSVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 7 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRLFLGDQWSVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 8 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTFLGDQWTVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 9 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTFLGDQWCVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 10 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVKQAAGPAFELYRTFLGDQWCAADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 11 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVKQAAGPAFELYRTFLGDQWCTADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 12 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRSFLGDQWCVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 13 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTFLGDQWCVADWDGFAAAAKASPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 14 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWDGFAAAAKASPSKIMSLFPSSDEPPQNLKEYDPEWARAFFEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 15 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 16 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERALAIIESAGVPVEYASFPDALHMMHQFEPERFAEVLTSWIARLPADTPGA

SEQ ID NO: 17 - protein, 316 aa, Artificial Sequence
  variant alpha/beta hydrolase
MTDQSPETRNIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 18 - protein, 316 aa, Artificial Sequence
  variant alpha/beta hydrolase
MTDQSPETRDIFVPHAYEEQQVDLGEVVMNYVEAGSADHPALLLLPEQTGSWWSYEPVMGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 19 - protein, 316 aa, Artificial Sequence
  variant alpha/beta hydrolase
MTDQSPETRDIFVPHAFEEQQVDLGEVTMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 20 - protein, 316 aa, Artificial Sequence
  variant alpha/beta hydrolase
MTDQSPETRDIFVPHAFEEQQVDLGEVVMNYVEAGSADRPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 21 - protein, 316 aa, Artificial Sequence
  variant alpha/beta hydrolase
MTDQSPETRDIFVPHAFEEQQVDLGEVVMNYVEAGSADHPALLLLPSQTESWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 22 - protein, 316 aa, Artificial Sequence
  variant alpha/beta hydrolase
MTDQSPETRDIFVPHAFEEQQVDLGEVVMNYVEAGSADHPALLLLPEQTGSWWSYEEAMGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 23 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLSEHFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 24 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVYAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 25 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYSMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 26 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDPPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 27 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALSCPHDLMLSQVKTPILITHHARAVDPHSGDLLGALSDFQAERAQAIIESAGVPVEYASFPDALHMMHQFEPERFAEVLTSWVARLPADTPGA

SEQ ID NO: 28 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDRMLSQVKTPILITHHARAVDPHSGDLLGALSDFQAERAQAIIESAGVPVEYASFPDALHMMHQFEPERFAEVLTSWVARLPADTPGA

SEQ ID NO: 29 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHVRAVDPHSGDLLGALSDFQAERAQAIIESAGVPVEYASFPDALHMMHQFEPERFAEVLTSWVARLPADTPGA

SEQ ID NO: 30 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPESGDLLGALSDFQAERAQAIIESAGVPVEYASFPDALHMMHQFEPERFAEVLTSWVARLPADTPGA

SEQ ID NO: 31 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYESFPDALHMMHQFEPERFAEVLTSWVARLPADTPGA

SEQ ID NO: 32 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDATHSMHQFEPERFAEVLTSWVARLPADTPGA

SEQ ID NO: 33 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQSEPERFAEVLTSWVARLPADTPGA

SEQ ID NO: 34 - protein, 316 aa, Artificial Sequence
  variant alpha/beta hydrolase
MTDQSPETRDIFVPHAFEEQQVDLGEVVMNYVEAGSADHPALLLLPEQTGSWWSYEPVIGLLAESFHVFAVDIRGQGRSTWTPKRYSLDNFGNDLVRFISLVVKRPVIVSGNSSGGVLAAWLSAYAMPGQIRAALCEDTPFFASELVPAHGHSVRQAAGPAFELYRTYLGDQWCVADWEGFCRAARRSPSKIMSLFPSSDEPPQNLKEYDPEWARAFYEGTVALHCPHDLMLSQVKTPILITHHARAVDPHSGDLLGALSDFQAERAQAIIESAGVPVEYASFPDALHMMHQFEPERFAEVLTRWVARLPADTPGA

SEQ ID NO: 35 - protein, 316 aa, Artificial Sequence
  variant alpha/beta hydrolase
MTDQSPETRNIFVPHAFEEQQVDLGEVTMNYVEAGSADRPALLLLPEQTGSWWSYEEAMGLLSEHFHVYAVDIRGQGRSTWTPKRYSLDNFGNDLVRFISLVVKRPVIVSGNSSGGVLAAWLSAYAMPGQIRAALCEDPPFFASELVPAHGHSVRQAAGPAFELYRTYLGDQWCVADWEGFCRAARRSPSKIMSLFPSSDEPPQNLKEYDPEWARAFYEGTVALSCPHDRMLSQVKTPILITHHARAVDPHSGDLLGALSDFQAERAQAIIESAGVPVEYESFPDATHSMHQSEPERFAEVLTRWVARLPADTPGA

SEQ ID NO: 36 - protein, 10 aa, Artificial Sequence
  consensus amino acid motif 1
  VARIANT (7)..(7): Xaa is Val or Ile
  VARIANT (8)..(8): Xaa is Val or Thr
QVDLGEXXMN

SEQ ID NO: 37 - protein, 15 aa, Artificial Sequence
  consensus amino acid motif 2
  VARIANT (7)..(7): Xaa is Gly or Ala
  VARIANT (9)..(9): Xaa is Ala or Val
  VARIANT (11)..(11): Xaa is Phe or Tyr
EYDPEWXRXFXEGTV

SEQ ID NO: 38 - protein, 14 aa, Artificial Sequence
  consensus amino acid motif 3
  VARIANT (3)..(3): Xaa is Ser or Gly
  VARIANT (9)..(9): Xaa is Ile or Val
  VARIANT (11)..(11): Xaa is Ile or Leu
MLXQVKTPXLXTHH

SEQ ID NO: 39 - protein, 127 aa, Artificial Sequence
  consensus amino acid motif 4
  VARIANT (3)..(3): Xaa is Val or Leu
  VARIANT (6)..(6): Xaa is Leu or Ile
  VARIANT (18)..(18): Xaa is Pro or Glu
  VARIANT (19)..(19): Xaa is Val or Ala
  VARIANT (20)..(20): Xaa is Ile or Met
  VARIANT (24)..(24): Xaa is Ala or Ser
  VARIANT (26)..(26): Xaa is Ser or Asn or His
  VARIANT (30)..(30): Xaa is Phe or Tyr
  VARIANT (34)..(34): Xaa is Ile or Leu
  VARIANT (41)..(41): Xaa is Thr or Ser
  VARIANT (45)..(45): Xaa is Lys or Arg
  VARIANT (61)..(61): Xaa is Ser or Ala or Asn
  VARIANT (64)..(64): Xaa is Val or Ile
  VARIANT (69)..(69): Xaa is Ile or Val
  VARIANT (71)..(71): Xaa is Ser or Ala
  VARIANT (78)..(78): Xaa is Val or Leu
  VARIANT (87)..(87): Xaa is Ala or Ser
  VARIANT (92)..(92): Xaa is Ile or Leu
  VARIANT (94)..(94): Xaa is Ala or Gly
  VARIANT (95)..(95): Xaa is Ala or Val
  VARIANT (100)..(100): Xaa is Thr or Ala or Pro
  VARIANT (101)..(101): Xaa is Pro or Ala
  VARIANT (111)..(111): Xaa is Tyr or His
  VARIANT (116)..(116): Xaa is Leu or Arg
  VARIANT (118)..(118): Xaa is Ala or Gly
  VARIANT (122)..(122): Xaa is Ala or Val
  VARIANT (126)..(126): Xaa is Tyr or Phe
PAXLLXPEQTGSWWSYEXXXGLLXEXFHVXAVDXRGQGRSXWTPXRYSLDNFGNDLVRFIXLVXKRPVXVXGNSSGGXLAAWLSAYXMPGQXRXXLCEDXXFFASELVPAXGHSVXQXAGPXFELXR